(19) United States
(12) Patent Application Publication
(10) Pub. No.: US 2013/0287856 A1
(43) Pub. Date: Oct. 31, 2013

(54) Drug Delivery Dispersion And Film Formed Therefrom

(71) Applicants: Virginie Caprasse, Oreye (BE); Fabrizio S. Galeone, Buvrinnes (BE); Leon Andre Marteaux, Bxl (Auderghem) (BE); Anke Sieg, Bruxelles (Ixelles) (BE); Xavier Jean-Paul Thomas, Famars (FR)

(72) Inventors: Virginie Caprasse, Oreye (BE); Fabrizio S. Galeone, Buvrinnes (BE); Leon Andre Marteaux, Bxl (Auderghem) (BE); Anke Sieg, Bruxelles (Ixelles) (BE); Xavier Jean-Paul Thomas, Famars (FR)

(21) Appl. No.: 13/976,312

(22) PCT Filed: Dec. 27, 2011
(86) PCT No.: PCT/US11/67400

§ 371 Date: Jun. 26, 2013

(30) Foreign Application Priority Data
Dec. 27, 2010 (EP) 10306521.5

(51) Int. Cl. A61K 9/107

(52) U.S. Cl. CPC A61K 9/1075
USPC 424/490

(57) ABSTRACT

An active agent delivery dispersion includes water, 1 to 98 weight percent of a plurality of encapsulated particles dispersed in the water, and 0.1 to 20 weight percent of an active agent, each based on a total weight of the dispersion. The active agent is dispersed in the water independently from the plurality of encapsulated particles. Each of the particles includes a core and a layer including a silica disposed about the core. The plurality includes first and second populations of encapsulated particles. The core of the first population includes a first organopolysiloxane having at least two silicon-bonded alkenyl groups per molecule and a hydro-silylation catalyst. The core of the second population includes an organohydrogensiloxane having an average of greater than two silicon bonded hydrogen atoms per molecule and a second organopolysiloxane having at least two silicon-bonded alkenyl groups per molecule.

### FIELD OF THE DISCLOSURE

The present disclosure generally relates to an active agent delivery dispersion including a plurality of encapsulated particles including first and second populations each having a core and layer including a silica disposed about the core.

### DESCRIPTION OF THE RELATED ART

Water-based hydrosilylation curable siloxane compositions are commonly used in many industrial processes. The compositions may be two component emulsion systems wherein a first emulsion contains a hydrosilylation catalyst and a vinyl terminated polydimethylsiloxane (PDMS) and a second emulsion contains an organohydrogensiloxane. When the first and second emulsions are combined, an undesirable ripening phenomena tends to occur because the components from the emulsions diffuse together resulting in premature and unwanted reaction and curing. To minimize this possibility, a cure inhibitor may be added to one or both of the emulsions to extend the “bath life” and minimize premature curing. These compositions may be cured by the evaporation of the cure inhibitor at high temperatures. The use of cure inhibitors increases the complexity and cost of the emulsions and increases the time needed to form the emulsions and cure the compositions. Accordingly, there remains an opportunity to develop improved curable siloxane compositions for use in aqueous applications.

### SUMMARY OF THE DISCLOSURE AND ADVANTAGES

The instant disclosure provides an active agent delivery dispersion. The dispersion includes water and 1 to 98 weight percent of a plurality of encapsulated particles dispersed in the water wherein the weight percent is based on a total weight of the dispersion. Each of the particles includes a core and a layer including a silica that is disposed about the core. The plurality includes a first population and a second population of encapsulated particles. The core of the first population of particles includes a first organopolysiloxane having at least two silicon-bonded alkenyl groups per molecule and a hydrosilylation catalyst. The core of the second population of particles includes an organohydrogensiloxane having an average of greater than two silicon bonded hydrogen atoms per molecule and a second organopolysiloxane having at least two silicon-bonded alkenyl groups per molecule that is the same or different from the first organopolysiloxane. The dispersion also includes 0.01 to 20 weight percent of an active agent dispersed in the water independently from the plurality of encapsulated particles wherein the weight percent is also based on a total weight of the dispersion. This disclosure also provides a film that includes a cured organopolysiloxane formed from the plurality of encapsulated particles and also includes 0.02 to 60 weight percent of the active agent dispersed in the cured organopolysiloxane.

The encapsulated particles allow the first and second organopolysiloxanes to be effectively combined and reacted with the organohydrogensiloxane in a controlled environment. The second organopolysiloxane in the second population of particles also balances the concentration of the silicon-bonded hydrogen atoms and the concentration of the hydrosilylation catalyst thereby minimizing premature reaction and curing and promoting a controlled and balanced cure. Furthermore, the encapsulated particles minimize a need to utilize cure inhibitors thereby reducing the cost and complexity of the dispersions. Moreover, the film delivers the active agent efficiently and effectively and at a predictable rate with minimized variance. The encapsulated particles also contribute to increased substantivity of the film and allow the film to be customized relative to occlusivity, permeability, and spreadability.

### BRIEF DESCRIPTION OF THE SEVERAL VIEWS OF THE DRAWINGS

Other advantages of the present disclosure will be readily appreciated, as the same becomes better understood by reference to the following detailed description when considered in connection with the accompanying drawings wherein:

FIG. 1A is a micrograph illustrating one encapsulated particle that includes a core and a silica layer disposed about the core;

FIG. 1B illustrates a magnified view of the micrograph of FIG. 1a;

FIG. 2A is a generalized schematic illustrating aggregation, coalescence, and breakage of encapsulated particles that allow the cores of the particles to react;

FIG. 2B is a micrograph illustrating agglomeration and coalescence of encapsulated particles;

FIG. 2C is a micrograph illustrating breakage of the encapsulated particles which allows the cores of the particles to react;

FIG. 3 is a line graph illustrating a percent extractable of unreacted organopolysiloxanes/organohydrogensiloxanes in various films of this disclosure as a function of time of curing reaction and ageing of encapsulated particles;

FIG. 4 is a bar graph showing vapor permeability of a first collagen membrane, an additional collagen membrane that includes an embodiment of the film of this disclosure disposed thereon, and two additional collagen membranes that each include comparative films disposed thereon;

FIG. 5 is a bar graph showing substantivity percentage as a function of a number of washes of two embodiments of the film of this disclosure and of two comparative films, evidenced by an amount of the films remaining on human skin as a function of a number of washes;

FIG. 6 is a line graph showing substantivity percentage as a function of time of one embodiment of the film of this disclosure as compared to a film formed from Kelo-cote, which is a commercially available product used for delivery of active agents on skin, as a function of time;

FIG. 7 is a graph of a sensory profile of one embodiment of the film of this disclosure as compared to a film formed from Kelo-cote, which is a commercially available product;

FIG. 8 is a line graph showing cumulative lidocaine penetration across a pig skin as a function of time from one embodiment of a film of this disclosure as compared to a film formed from EMLA, which is a commercially available product used for delivery of lidocaine into skin; and

FIG. 9 is a line graph showing cumulative caffeine penetration across a pig skin as a function of time from one embodiment of a film of this disclosure as compared to a film formed from Nuxe and a second film formed from Elancyl, both of which are commercially available products used for delivery of caffeine into skin.

### DETAILED DESCRIPTION OF THE DISCLOSURE

The present disclosure provides an active agent delivery dispersion (hereinafter referred to as the “dispersion”) and a film formed from the dispersion. The active agent in the dispersion and the film are each described in greater detail below.

The dispersion includes water and a plurality of encapsulated particles dispersed in the water. If the encapsulated particles are described as solids dispersed in the water, then the dispersion may be further defined as a sol, suspension, gel, or colloidal solution. Colloidal solutions tend to include particles of less than 100 nanometers in size dispersed in the continuous phase. If the encapsulated particles are described as liquids, then the dispersion may be further defined as an emulsion such an oil in water (O/W) emulsion, water in oil (W/O) emulsion, water in oil in water (W/O/W) emulsion, ionic or nonionic emulsion, anionic, cationic, or amphoteric emulsion, microemulsion, miniemulsion, multiple emulsion, artificial emulsion, and the like.

The water of the dispersion may be tap water, well water, purified water, deionized water, and combinations thereof and may be present in the dispersion in varying amounts depending on the type of dispersion. The water may be the continuous phase and the plurality of encapsulated particles may be the dispersed phase. In various embodiments, the water is present in amounts of from 1 to 99, of from 5 to 95, 10 to 90, to 85, 20 to 80, 25 to 75, 30 to 70, 35 to 65, 40 to 60, 45 to 55, from 5 to 70, from 10 to 70, from 20 to 70, from 30 to 70, from 40 to 70, from 50 to 70, or from 60 to 70, or about 50, parts by weight per 100 parts by weight of the dispersion. Alternatively, the water is present as a balance of the dispersion that includes the plurality of encapsulated particles and the active agent.

It is also contemplated that one or more supplementary solvents may be combined with the water. The supplemental solvents may be hydrophilic and polar and may include alcohols, solvents that include —OH groups, ethers, esters, and the like. Further, the water may be combined with one or more drug delivery enhancers (such as propylene glycol and pentylene glycol), occlusive agents (such as petrolatum and mineral oil), or any of the additives, surfactants, or other components described in greater detail below.

### Plurality of Encapsulated Particles

The dispersion includes from 1 to 98 weight percent of the encapsulated particles wherein this weight percent represents the total weight of the following in the dispersion: (a) first organopolysiloxane having at least two silicon-bonded alkenyl groups per molecule, a (b) hydrosilylation catalyst, a (c) organohydrogensiloxane having an average of greater than two silicon bonded hydrogen atoms per molecule, a (d) second organopolysiloxane having at least two silicon-bonded alkenyl groups per molecule that is the same or different from the (a) first organopolysiloxane, and a layer including the silica, each of which is described in greater detail below. In another embodiment, the dispersion includes 1 to 80 weight percent of the encapsulated particles. In still other embodiments, the dispersion includes from 5 to 70, from 10 to 70, from 30 to 70, from 15 to 65, from 20 to 60, from 25 to 55, from 30 to 50, from 35 to 45, or from 25 to 35, weight percent of the encapsulated particles (i.e., the weight percent of (a)-(d)) based on a total weight of the dispersion. Alternatively, the dispersion includes from 70 to 90, from 75 to 90 from 80 to 90, from 85 to 90, from 75 to 85, from 80 to 85, from 5 to 75, or of about 25, of about 30, of about 35, of about 40, or of about 45, weight percent of the encapsulated particles (i.e., the weight percent of (a)-(d)) based on a total weight of the dispersion.

Each of the encapsulated particles is not particularly limited in size (e.g. diameter) and the encapsulated particles that make up the plurality may be present in a distribution of various sizes. The encapsulated particles have a size distribution reported as Dᵥ(0.5) of from 0.2 to 500, from 0.5 to 500, from 1 to 450, from 5 to 400, from 50 to 350, from 100 to 300, from 150 to 250, or from 200 to 250, micrometers, as well defined and appreciated in the art. In other embodiments, the encapsulated particles have a size distribution reported as Dᵥ(0.1) of from 0.025 to 1, from 0.05 to 1, from 0.1 to 1, from 0.1 to 0.9, from 0.2 to 0.8, from 0.3 to 0.7, from 0.4 to 0.6, or from 0.4 to 0.5, micrometers. In still other embodiments, the encapsulated particles have a size distribution reported as Dᵥ(0.9) of from 1 to 1000, from 50 to 950, from 100 to 900, from 150 to 850, from 200 to 800, from 250 to 750, from 300 to 700, from 350 to 650, from 400 to 600, from 450 to 550, or from 450 to 500, micrometers. In even further embodiments, the encapsulated particles have a Dᵥ(0.1) of from 0.05 to 0.75, a Dᵥ(0.5) of from 0.5 to 100, and/or a Dᵥ(0.9) of from 2 to 750, micrometers. The size of the encapsulated particles may be measured using a laser diffraction particle size analyzer such as a Malvern Mastersizer.

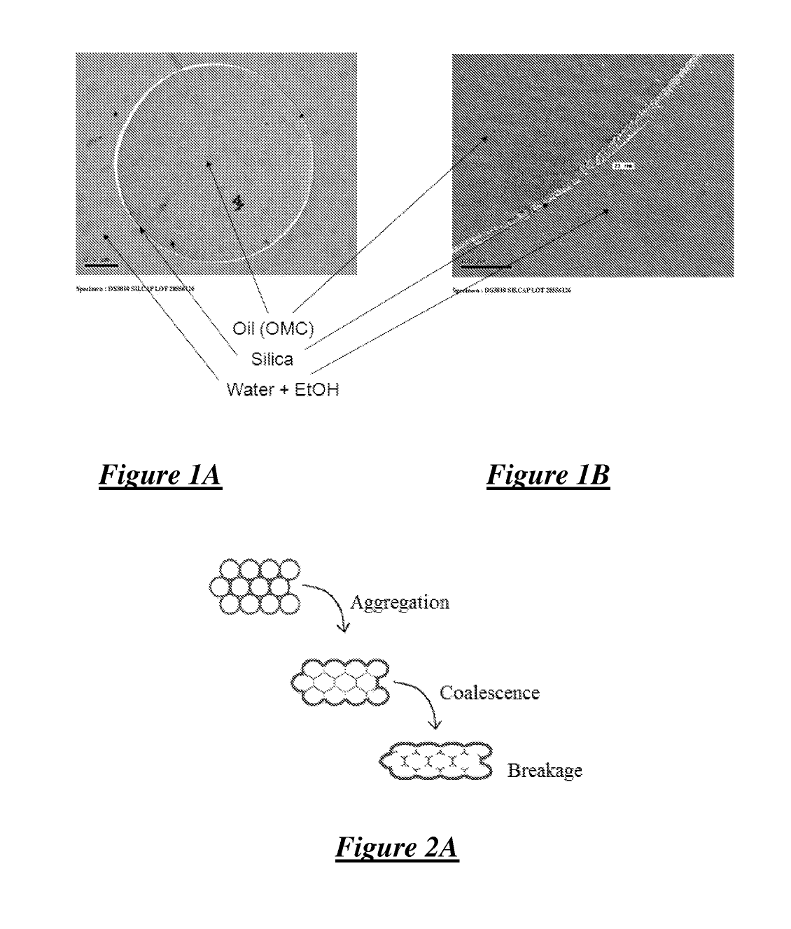

Each of the encapsulated particles includes a core and a layer disposed about (e.g. around) the core as shown in FIGS. 1A and 1B. In these Figures, the core is labeled as “oil” and the layer disposed about the core is labeled as “silica.” The terminology “disposed about” includes the layer disposed around all or a portion of the core. The layer is usually continuous but may be discontinuous at points. The layer includes a silica such as silicon dioxide (SiO₂) (traditionally known as “silica”) or an organo-modified silica (traditionally known as Ormosils) or a silica hybrid. Suitable examples of organo-modified silicas and/or silica hybrids include, but are not limited to, compounds having the general formula [RSi_{1.75}O₃]ₙ or [R₂Si₂O₃]ₙ where R is an organic group and n is an number of at least one. In one embodiment, the silica is formed from a hydrolysis/condensation reaction of tetraethylorthosilicate (TEOS) and water to form silica (SiO₂) and C₂H₅OH.

The plurality of encapsulated particles includes a first population and a second population. In the first population, the core of the encapsulated particles includes an (a) first organopolysiloxane having at least two silicon-bonded alkenyl groups per molecule and a (b) hydrosilylation catalyst. In the second population, the core of the encapsulated particles includes (c) organohydrogensiloxane having an average of greater than two silicon bonded hydrogen atoms per molecule and (d) a second organopolysiloxane having at least two silicon-bonded alkenyl groups per molecule that is the same or different from the (a) first organopolysiloxane. Each of (a)-(d) is described in greater detail below. Additionally, it is contemplated that any one or more of (a)-(d) and any additives or additional compounds included in the dispersion, cores, layers, etc. of this disclosure may be the same or different that those described in PCT Ser. No. PCT/US10/40124 filed on Jun. 28, 2010, which is expressly incorporated herein by reference in its entirety.

### (a) First Organopolysiloxane—At Least Two Silicon-Bonded Alkenyl Groups Per Molecule

Organopolysiloxanes are polymers including siloxy units independently selected from (R₃SiO_{1/2}), (R₂SiO_{2/2}), (RSiO_{3/2}), or (SiO_{4/2}) siloxy units, where R may be a hydrocarbon group. These siloxy units can be combined in various manners to form cyclic, linear, or branched structures. The chemical and physical properties of the resulting polymeric structures can vary. For example organopolysiloxanes can be volatile fluids, low viscosity fluids, high viscosity fluids/gums, elastomers, rubbers, or resins.

The (a) first organopolysiloxane having at least two silicon-bonded alkenyl groups per molecule may be selected from any organopolysiloxane, or mixture of organopolysiloxanes including at least two siloxy units represented by the formula R²RₘSiO_{(4-m)/2} wherein R is independently a hydrocarbon group having from 1 to 20 carbon atoms, each R² is a monovalent alkenyl group, e.g. having from 2 to 12 carbon atoms, and m is a number of from 0 to 2. The R² alkenyl groups of the (a) first organopolysiloxane having at least two silicon-bonded alkenyl groups per molecule are exemplified by vinyl, allyl, 3-butenyl, 4-pentenyl, 5-hexenyl, 6-heptenyl, 7-octenyl, 8-nonenyl, 9-decenyl, 10-undecenyl, 4,7-octadienyl, 5,8-nonadienyl, 5,9-decadienyl, 6,11-dodecadienyl and 4,8-nonadienyl. The R² alkenyl group may be present on any mono, di, or tri siloxy unit in the organopolysiloxane, for example, (R²R₂SiO_{1/2}), (R²RSiO_{2/2}), or (R²SiO_{3/2}), as well as in combination with other siloxy units not including an R² substituent, such as (R₃SiO_{1/2}), (R₂SiO_{2/2}), (RSiO_{3/2}), or (SiO_{4/2}) siloxy units where R is a hydrocarbon including 1 to 20 carbons, alternatively an alkyl group including 1 to 12 carbons, alternatively an alkyl group including 1 to 6 carbons or alternatively methyl providing there are at least two R² substituents in the organopolysiloxane. The monovalent hydrocarbon group R having from 1 to 20 carbon atoms is exemplified by alkyl groups such as methyl, ethyl, propyl, butyl, hexyl, octyl, and decyl, cycloaliphatic groups such as cyclohexyl, aryl groups such as phenyl, tolyl, and xylyl, and aralkyl groups such as benzyl and phenylethyl.

Representative, non-limiting, examples of such organopolysiloxanes suitable as the (a) first organopolysiloxane having at least two silicon-bonded alkenyl groups per molecule include those having the average formula (R₂R²SiO_{1/2})ᵥ(R₂SiO_{2/2})ₓ, (R₂R²SiO_{1/2})ᵥ(R₂SiO_{2/2})ₓ(R²RSiO_{2/2})_{y}, (R₂R²SiO_{1/2})ᵥ(R₂SiO_{2/2})ₓ(RSiO_{3/2})_{z}, (R₂R²SiO_{1/2})ᵥ(R₂SiO_{2/2})ₓ (RSiO_{3/2})_{z}(SiO_{4/2})_{w}, (R₂R²SiO_{1/2})ᵥ(SiO₂)_{w}(R₂SiO)ₓ, (R₃SiO_{1/2})ᵥ(R₂SiO)ₓ(R²RSiO_{2/2})_{y}, (R₃SiO_{1/2})ᵥ (R₂SiO)ₓ(R²RSiO)_{y}, (R₃SiO_{1/2})ᵥ(R₂SiO)ₓ(R²RSiO)_{y}(RSiO_{3/2})_{z}, (R₃SiO_{1/2})ᵥ(R₂SiO)ₓ(R²RSiO)_{y} (SiO₂)_{w}, (R₃SiO_{1/2})ᵥ(R₂SiO)ₓ (R²RSiO)_{y}(SiO₂)_{w}(RSiO_{3/2})_{z}, and/or (R₃SiO_{1/2})ᵥ(R₂SiO)ₓ(R²SiO_{3/2})_{z}, where v≧2, w≧0, x≧0, y≧2, and z is ≧0, and wherein R and R² are as described above.

The (a) first organopolysiloxane may also be or include a mixture of any of the aforementioned organopolysiloxanes. The molecular weight of the (a) first organopolysiloxane may vary, and is not limiting. However, when molecular weights become too high, or if the (a) first organopolysiloxane is a solid, it may be difficult to handle or incorporate the (a) first organopolysiloxane in the encapsulated particles. Thus, it may be desirable to dilute the (a) first organopolysiloxane in a suitable solvent or lower molecular weight fluid, such as a less viscous silicone fluid. The viscosity of the (a) first organopolysiloxane or dispersion of the (a) first organopolysiloxane in the lower molecular weight fluid may vary from 1 to 10,000 mPa·s, alternatively, 50 to 1000 mPa·s, or alternatively, 100 to 1000 mPa·s, when measured at 25° C.

In various embodiments, the (a) first organopolysiloxane is be selected from the group consisting of trimethylsiloxy-terminated polydimethylsiloxane-polymethylvinylsiloxane copolymers, vinyldimethylsiloxy-terminated polydimethylsiloxane-polymethylvinylsiloxane copolymers, trimethylsiloxy-terminated polydimethylsiloxane-polymethylhexenylsiloxane copolymers, hexenyldimethylsiloxy-terminated polydimethylsiloxane-polymethylhexenyl siloxane copolymers, trimethylsiloxy-terminated polymethylvinylsiloxane polymers, trimethylsiloxy-terminated polymethylhexenylsiloxane polymers, vinyldimethylsiloxy-terminated polydimethylsiloxane polymers, and hexenyldimethylsiloxy-terminated polydimethylsiloxane polymers, each having a degree of polymerization of from 10 to 300, or alternatively having a viscosity at 25° C. of 10 to 1000 mPa·s.

Alternatively the (a) first organopolysiloxane may be selected from vinyl functional endblocked polydimethylsiloxanes (vinyl siloxanes) or hexenyl functional endblocked polydimethylsiloxanes (hexenyl siloxanes), such as those having the average formula CH₂═CH(Me)₂SiO[Me₂SiO]_{x′}Si(Me)₂CH═CH₂, CH₂═CH—(CH₂)₄-(Me)₂SiO[Me₂SiO]_{x′}Si(Me)₂-(CH₂)₄—CH═CH₂, or Me₃SiO[(Me)₂SiO]_{x′}[CH₂═CH(Me)SiO]_{x″}SiMe₃, wherein Me is methyl, x′≧0, alternatively x is 0 to 200, alternatively x is 10 to 150, x″≧2, alternatively x″ is 2 to 50, and alternatively x″ is 2 to 10.

Vinyl or hexenyl functional polydimethylsiloxanes may be used and non-limiting examples include DOW CORNING® fluids, SFD 128, DC4-2764, DC2-7891, DC2-7754, DC2-7891, and DC2-7463, SFD-117, SFD-119, SFD 120, SFD 129, DC 5-8709, LV, 2-7038, DC2-7892, 2-7287, 2-7463, and dihexenyl terminal DC7692, DC7697 (Dow Corning Corporation, Midland, Mich.).

The (a) first organopolysiloxane may be included in the core of the first population in amounts as described in PCT Ser. No. PCT/US10/40124 filed on Jun. 28, 2010, which is expressly incorporated herein by reference in its entirety.

### (b) Hydrosilylation Catalyst

The (b) hydrosilylation catalyst may be any suitable Group VIII metal based catalyst selected from a platinum, rhodium, iridium, palladium, and/or ruthenium. Group VIII group metal including catalysts useful in this disclosure can be any of those known to catalyze reactions of silicon bonded hydrogen atoms with silicon bonded unsaturated hydrocarbon groups, e.g. in hydrosilylation reaction. The preferred Group VIII metal for use in this disclosure is a platinum based catalyst. Some preferred platinum based catalysts include, but are not limited to, platinum metal, platinum compounds and platinum complexes.

Non-limiting examples of suitable (b) hydrosilylation catalysts are described in U.S. Pat. No. 2,823,218 (commonly referred to as “Speier's catalyst) and U.S. Pat. No. 3,923,705, expressly incorporated herein by reference. The (b) hydrosilylation catalyst may be a “Karstedt's catalyst”, which is described in U.S. Pat. Nos. 3,715,334 and 3,814,730, expressly incorporated herein by reference. Karstedt's catalyst is a platinum divinyl tetramethyl disiloxane complex typically including about one-weight percent of platinum in a solvent such as toluene. Alternatively the (b) hydrosilylation catalyst may include or be a reaction product of chloroplatinic acid and an organosilicon compound including terminal aliphatic unsaturation, as described in U.S. Pat. No. 3,419,593, expressly incorporated herein by reference. Alternatively, the (b) hydrosilylation catalyst may include a neutralized complex of platinum chloride and divinyl tetramethyl disiloxane, as described in U.S. Pat. No. 5,175,325, also incorporated herein by reference. Alternatively, the (b) hydrosilylation catalyst may be as described in PCT Ser. No. PCT/US10/40124 filed on Jun. 28, 2010, which is expressly incorporated herein by reference in its entirety.

Additional suitable, but non-limiting, (b) hydrosilylation catalysts include rhodium catalysts such as [Rh(O₂CCH₃)₂]₂, Rh(O₂CCH₃)₃, Rh₂(C₈H₁₅O₂)₄, Rh(C₅H₇O₂)₃, Rh(C₅H₇O₂)(CO)₂, Rh(CO)[Ph₃P](C₅H₇O₂), RhX⁴₃[(R³)₂S]₃, (R²₃P)₂Rh(CO)X⁴, (R²₃P)₂Rh(CO)H, Rh₂X⁴₂Y²₄, HₐRh_{b}olefin_{c}Cl_{d}, Rh (O(CO)R³)₃₋ₙ(OH)ₙ wherein X⁴ is hydrogen, chlorine, bromine or iodine, Y² is an alkyl group, such as methyl or ethyl, CO, C₈H₁₄ or 0.5 C₈H₁₂, R³ is an alkyl radical, cycloalkyl radical or aryl radical and R² is an alkyl radical an aryl radical or an oxygen substituted radical, a is 0 or 1, b is 1 or 2, c is a whole number from 1 to 4 inclusive and d is 2, 3 or 4, n is 0 or 1. Any suitable iridium catalysts such as Ir(OOCCH₃)₃, Ir(C₅H₇O₂)₃, [Ir(Z⁴)(En)₂]₂, or (Ir(Z⁴)(Dien)]₂, where Z⁴ is chlorine, bromine, iodine, or alkoxy, En is an olefin and Dien is cyclooctadiene may also be used. Still other suitable (b) hydrosilylation catalyst include are described in U.S. Pat. Nos. 3,159,601, 3,220,972, 3,296,291, 3,516,946, 3,989,668, 4,784,879, 5,036,117, and 5,175,325 and EP 0 347 895 B, each of which is expressly incorporated herein by reference.

The (b) hydrosilylation catalyst may be utilized in the core of the first population in amounts of 0.001 or greater parts by weight of elemental platinum group metal, per one million parts (ppm) of the cores of the first and second populations combined. The concentration of the (b) hydrosilylation catalyst may be capable of providing an equivalent of at least 1 part per million of elemental platinum group metal. A concentration providing the equivalent of 1 to 500, alternatively 50 to 500, alternatively 50 to 200 parts per million of elemental platinum may also be used. In one embodiment, the (a) organopolysiloxane is present in an amount of from 96-98 weight percent of a total weight of the core of the encapsulated particles of the first population while the (b) hydrosilylation catalyst is present in an amount of about 79 ppm in a film.

### (c) Organohydrogensiloxane—Average of Greater Than Two Silicon Bonded Hydrogen Atoms Per Molecule

Organohydrogensiloxanes are organopolysiloxanes having at least one SiH including siloxy unit, that is at least one siloxy unit in the organopolysiloxane has the formula (R₂HSiO_{1/2}), (RHSiO_{2/2}), or (HSiO_{3/2}). The (c) organohydrogensiloxane having an average of greater than two silicon bonded hydrogen atoms per molecule is not particularly limited and may include any organopolysiloxane including a silicon-bonded hydrogen atom (SiH). Thus, the (c) organohydrogensiloxane may include any number of (R₃SiO_{1/2}), (R₂SiO_{2/2}), (RSiO_{3/2}), (R₂HSiO_{1/2}), (RHSiO_{2/2}), (HSiO_{3/2}) or (SiO_{4/2}) siloxy units, providing there are on average at least two SiH siloxy units in the molecule. The (c) organohydrogensiloxane can include or be a single linear or branched organohydrogensiloxane or a combination including two or more linear or branched organohydrogensiloxanes that differ in at least one of structure, viscosity, average molecular weight, siloxane units, and/or sequence. Although not particularly limited, the viscosity of the (c) organohydrogensiloxane is may be of from 3 to 10,000 mPa·s, alternatively from 3 to 1,000 mPa·s, or alternatively from 10 to 500 mPa·s, when measured at 25° C.

The amount of SiH units present in the (c) organohydrogensiloxane may vary, providing there are at least two SiH units per molecule. The amount of SiH units present in the (c) organohydrogensiloxane is expressed herein as % SiH which is the weight percent of hydrogen in the (c) organohydrogensiloxane. The % SiH may vary from 0.01 to 10%, alternatively from 0.1 to 5%, or alternatively from 0.5 to 2%.

In various embodiments, the (c) organohydrogensiloxane has the average formula, (R³₃SiO_{1/2})ₐ(R⁴₂SiO_{2/2})_{b}(R⁴HSiO_{2/2})_{c} wherein R³ is hydrogen or R⁴, R⁴ is a monovalent hydrocarbon group having from 1 to 10 carbon atoms, a≧2, b≧0, alternatively b=1 to 500, alternatively b=1 to 200, c≧2, alternatively c=2 to 200, alternatively c=2 to 100. R⁴ may be a substituted or unsubstituted aliphatic or aromatic hydrocarbyl. Monovalent unsubstituted aliphatic hydrocarbyls are exemplified by, but not limited to, alkyl groups such as methyl, ethyl, propyl, pentyl, octyl, undecyl, and octadecyl and cycloalkyl groups such as cyclohexyl. Monovalent substituted aliphatic hydrocarbyls are exemplified by, but not limited to, halogenated alkyl groups such as chloromethyl, 3-chloropropyl, and 3,3,3-trifluoropropyl. The aromatic hydrocarbon group is exemplified by, but not limited to, phenyl, tolyl, xylyl, benzyl, styryl, and 2-phenylethyl.

In other embodiments, the (c) organohydrogensiloxane may include additional siloxy units and have the average formula (R³₃SiO_{1/2})ₐ(R⁴₂SiO_{2/2})_{b}(R⁴HSiO_{2/2})_{c}(R⁴SiO_{3/2})_{d}, (R³₃SiO_{1/2})ₐ(R⁴₂SiO_{2/2})_{b} (R⁴HSiO_{2/2})_{c}(SiO_{4/2})_{d}, (R³₃SiO_{1/2})ₐ(R⁴₂SiO_{2/2})_{b}(R⁴HSiO_{2/2})_{c}(SiO_{4/2})_{d}(R⁴SiO_{3/2})ₑ, or any mixture thereof, where each R³ is independently a hydrogen atom or R⁴, each R⁴ is independently a monovalent hydrocarbyl, e.g. having 1 to 10 carbon atoms, and a≧2, b≧0, c≧2, d≧0, and e is ≧0. In another embodiment, the (c) organohydrogensiloxane is selected from a dimethyl, methyl-hydrogen polysiloxane having the average formula, (CH₃)₃SiO[(CH₃)₂SiO]_{b} [(CH₃)HSiO]_{c}Si(CH₃)₃ where b≧0, alternatively, b=1 to 200, alternatively b=1 to 100, and c≧2, alternatively, c=2 to 100, alternatively c=2 to 50.

The (c) organohydrogensiloxane may be included in the core of the first population in amounts as described in PCT Ser. No. PCT/US10/40124 filed on Jun. 28, 2010, which is expressly incorporated herein by reference in its entirety.

### (d) Second Organopolysiloxane

Referring now to the (d) second organopolysiloxane, this organopolysiloxane has at least two silicon-bonded alkenyl groups per molecule and may be the same or different from the (a) first organopolysiloxane. In other words, the (d) second organopolysiloxane may be any one of those described above or may be different. The (d) second organopolysiloxane may be included in the core of the first in amounts as described in PCT Ser. No. PCT/US10/40124 filed on Jun. 28, 2010, which is expressly incorporated herein by reference in its entirety.

In one embodiment, [the (a) first organopolysiloxane and/or the (d) second organopolysiloxane] and the (c) organohydrogensiloxane are present in the dispersion in a molar ratio of SiH: alkenyl groups that varies from 3 to 10. In other embodiments, the amounts of [the (a) first organopolysiloxane and/or the (d) second organopolysiloxane] and the (c) organohydrogensiloxane in the dispersion may be adjusted to achieve a desired molar ratio of SiH: alkenyl groups to be greater than 1, alternatively in the range of 1 to 10, alternatively 1 to 4, alternatively 2 to 3.

In one embodiment, the (d) second organopolysiloxane is present in an amount of from 50 to 94 weight percent while the (c) organohydrogensiloxane is present in an amount of from 6 to 50 weight percent, of the core of the encapsulated particles of the second population. In another embodiment, the (d) second organopolysiloxane and the (c) organohydrogensiloxane provide a molar ratio of SiH/alkenyl groups that varies from 3 to 10, or alternatively from 4 to 9, or alternatively from 5 to 7. These ratios tend to promote curing as thin films provide adequate storage stability in dispersions.

While not intending to be bound by any particular theory, it is believed that the amounts of the (d) second organopolysiloxane and the (c) organohydrogensiloxane described immediately above promote partial reactions and promote more efficient overall curing. It is theorized that small amounts of the (b) hydrosilylation catalyst may permeate through the encapsulated particles of the second population and catalyze some reactions between the (d) second organopolysiloxane and the (c) organohydrogensiloxane.

It is also contemplated that the presence of colloidal silicate particles in the dispersion may limit the storage stability of the dispersion. Such colloid silicate particles may be formed as a side product when reacting water-reactive silicone compounds such as tetraalkoxysilanes. The storage stability may be improved by reducing the amount of colloidal silicate particles, or alternatively, by rendering the colloidal silicate particles non-reactive by addition of a colloidal silicate sequestering agent. As used herein “a colloidal silicate sequestering agent” may include any compound that interacts with the colloidal silicate particles in such a manner so as to minimize or prevent their reaction or coagulation. The colloidal silicate sequestering agent may be an organofunctional silane.

In one embodiment, the organofunctional silane is a quaternary functional trialkoxysilane. Representative, non-limiting examples of suitable quaternary functional trialkoxysilanes include Dow Corning® Q9-6346—Cetrimoniumpropyltrimethoxysilane Chloride. The colloidal silicate sequestering agent may be a silicone polyether. Silicone polyethers are commercially available. Representative, non-limiting examples of suitable silicone polyethers include Dow Corning® 190, 193, and 2-5657. Techniques for removing colloidal silicate particles and various colloidal silicate sequestering agents are further disclosed in U.S. App. Pub. No. 61/096,397, which is expressly incorporated herein by reference.

### Method of Forming the Plurality of Encapsulated Particles

The plurality of encapsulated particles may be formed by any method known in the art. In one embodiment, a sol-gel process (i.e., an in-situ polymerization process) is utilized wherein a silica or silicate precursor is combined with an oil. Representative, non-limiting examples of the in-situ process are those described in U.S. Pat. Nos. 6,159,453, 6,238,650, and 6,303,149, and WO 2005/009604, each of which is expressly incorporated herein by reference. In another embodiment, an ex-situ process is utilized wherein a silica or silicate precursor is polymerized in an emulsion polymerization process. Representative, non-limiting examples of such processes are described in WO03/066209, expressly incorporated herein by reference. Additional methods are described in PCT Ser. No. PCT/US10/40124 filed on Jun. 28, 2010, which is expressly incorporated herein by reference in its entirety.

In one embodiment, the plurality of encapsulated particles is prepared using a method that includes the following steps

I) forming an oil phase including the (a) organopolysiloxane, (b) hydrosilylation catalyst, (c) organohydrogensiloxane, and/or (d) second organopolysiloxane and combining the oil phase with an aqueous phase (e.g. solution) including a cationic or amphoteric surfactant to form an oil in water emulsion,

II) adding a water-reactive silicon compound to the oil in water emulsion wherein the water-reactive silicon compound includes, for example, a tetraalkoxysilane, and

III) polymerizing the water-reactive silicon compound at an oil/water interface of the oil in water emulsion to form particles of the first and/or second populations including the core and the layer disposed about the core.

The aforementioned embodiments of the method may be utilized once or more than once to form the plurality of encapsulated particles of the first and/or second populations. After formation, the first and second populations of the plurality of encapsulated particles may then combined with each other to form the dispersion of this disclosure.

Relative to Step (I), the oil phase and aqueous solution of the cationic or amphoteric surfactant may be mixed together to form an oil in water emulsion that is different from the dispersion of this disclosure that includes the water and the plurality of encapsulated particles described above. Mixing and emulsion formation may occur using any known techniques in the emulsion art. The oil phase and aqueous solution may be combined using simple stifling techniques. Particle size of the oil in water emulsion may then be reduced before addition of the water-reactive silicon compound by any emulsification device known in the art. Useful emulsification devices include, but are not limited to, homogenizers, sonolators, rotor-stator turbines, colloid mills, microfluidizers, blades, helices, and combination thereof. The particle size of the oil in water emulsion may range from 0.2 to 500 micrometers or from 0.5 micrometers and 100 micrometers.

The weight ratio of the oil phase to the aqueous phase may be between 40:1 and 1:50. Alternatively, the weight ratio of the oil phase to the aqueous phase is between 2:1 and 1:3. A phase inversion process can also be used in which the oil phase is mixed with a surfactant and a small amount of water, for example 2.5 to 10% by weight based on the oil phase, forming a water-in-oil emulsion which inverts to an oil-in-water emulsion upon shearing. Additional water can then be added for dilution. In one embodiment, the density of the oil phase and the density of the aqueous phase are approximately the same, i.e., the densities are “matched.” Alternatively, these densities can be within 2%, 1%, or 0.5% of each other.

Relative to Steps (II) and (III), the water-reactive silicon compound may include one or more alkoxy groups and each alkoxy group may include 1 to 4 carbons and alternatively 1 to 2 carbons. In one embodiment, the water-reactive silicon compound is further defined as a tetralkoxysilane such as tetraethoxysilane (TEOS) which may be utilized in monomeric form or as a liquid partial condensate or oligomer. Alkyl and alkoxy groups of the tetralkoxysilane may include from 1 to 4 carbon atoms or from 1 to 2 carbon atoms. The tetralkoxysilane may hydrolyze and form a network polymer that is a 3-dimensional network of silicone materials around emulsified droplets of one or more of (a), (b), (c), and/or (d).

It is contemplated that the tetraalkoxysilane can be used in conjunction with one or more water-reactive silicon compounds having at least two, alternatively at least 3, Si—OH groups or hydrolysable groups bonded to silicon (e.g. alkoxy or acyloxy groups bonded to silicon). Non-limiting examples of suitable water-reactive silicon compounds include alkyltrialkoxysilanes (e.g. methyltrimethoxysilane) or liquid condensates/oligomers thereof. Examples of suitable hydrolysable groups include alkoxy and acyloxy groups bonded to silicon atoms.

The water-reactive silicon compounds can include 50-100% by weight tetraalkoxysilane and 0-50% trialkoxysilane. Alternatively, the water-reactive silicon compounds may include at least 75% or alternatively 90 to 100% tetraalkoxysilane. In other embodiments, the water-reactive silicon compound includes an alkoxysilane having organofunctional groups such as a quaternized substituted alkyl groups. One typical quaternary alkoxysilane has the formula (CH₃O)₃SiCH₂CH₂CH₂N⁺(CH₃)₂(CH₂)₁₇CH₃Cl⁻.

The water-reactive silicon compound may be added to the oil-in-water emulsion as an undiluted liquid or as a solution in an organic solvent or in an emulsion. The water-reactive silicon compound and the oil-in-water emulsion may be combined or mixed during addition. In various embodiments, the amounts of tetraalkoxysilane in the water-reactive silicon compounds range from 6/1 to 1/13, alternatively from 1.2/1 to 1/7.3, alternatively from 1.3 to 1/6.1, based on the weight of the oil phase of the emulsion.

The tetraalkoxysilane and/or water-reactive silicon compounds may polymerize at the oil/water interface of the emulsion via a condensation reaction which may occur at acidic, neutral or basic pH. The condensation reaction generally occurs at ambient temperature and pressure, but can occur at increased temperature, for example up to 95° C., and increased or decreased pressure, for example under vacuum to strip volatile alcohols produced therein. In various embodiments, step (III) may be further defined as an “ex-situ emulsion polymerization” step wherein a tetraalkoxysilane precursor hydrolyzes and condenses at an oil/water interface leading to the formation of encapsulated particles via phase transfer.

It is contemplated that any catalyst known to promote the polymerization of the water-reactive silicon compound may be added during Step (III) to form the layer disposed about the core. The catalyst may be an oil soluble organic metal compound, for example an organic tin compound, particularly an organotin compound such as a diorganotin diester, for example dimethyl tin di(neodecanoate), dibutyl tin dilaurate or dibutyl tin diacetate, or alternatively a tin carboxylate such as stannous octoate, or an organic titanium compound such as tetrabutyl titanate. An organotin catalyst can, for example, be used at 0.05 to 2% by weight based on the water-reactive silicon compound. An organotin catalyst has the advantage of effective catalysis at neutral pH. The catalyst may be mixed before emulsification to promote condensation of the water-reactive silicon compound at the surface of emulsified droplets. The catalyst can alternatively be utilized before addition of the water-reactive silicon compound, simultaneously with the water-reactive silicon compound, or after the addition of the water-reactive silicon compound to harden and make more impervious the layer. Encapsulation of the core can however be achieved without catalyst. The catalyst, when used, can be added undiluted, or as a solution in an organic solvent such as a hydrocarbon, alcohol or ketone, or as a multi-phase system such as an emulsion or suspension.

In an alternative embodiment, the method includes the steps of:

A. forming a first oil phase comprising the (a) organopolysiloxane, and (b) hydrosilylation catalyst,

B. combining the oil phase with an aqueous phase comprising a surfactant to form an oil in water emulsion having an oil/water interface,

C. adding a tetraalkoxysilane to the oil in water emulsion,

D. polymerizing the tetraalkoxysilane at the oil/water interface of the emulsion to form the first population of encapsulated particles wherein the layer disposed about the core of the first population of encapsulated particles is silica,

E. forming a second oil phase comprising the (c) organohydrogensiloxane, and (d) second organopolysiloxane,

F. repeating steps B-D to form the second population of encapsulated particles disposed about the core of the second population of encapsulated particles is silica, and

G. combining the first and second populations of the encapsulated particles to form the dispersion.

One or more of the steps (A)-(G) may be the same as or similar to one or more of the steps (I)-(III) described above. Accordingly, any and all of the options described above for steps (I)-(III) may also be applied to one or more of the steps (A)-(G). One or more cationic surfactants, amphoteric surfactants, non-ionic surfactants, and/or other additives may be utilized as described in U.S. Pat. No. 5,035,832, and PCT Ser. No. PCT/US10/40124 filed on Jun. 28, 2010, each of which is expressly incorporated herein by reference in its entirety.

### Active Agent

Referring back to the active agent, the terminology “active agent” is not particularly limited and may refer to a pharmaceutically active agent, such as a drug, therapeutic agent, etc. In one embodiment, the active agent delivery dispersion is alternatively described as a drug delivery dispersion. The active agent may be hydrophilic or lipophilic and may be further defined as a hydrophilic drug or a lipophilic drug. In one embodiment, the active agent is further defined as a medicine, medication or medicament and may include any chemical substance intended for use in the medical diagnosis, cure, treatment, or prevention of disease. Alternatively, the active agent is further defined as a drug that may be administered transdermally on skin (e.g. mammalian or human skin). However, it is to be appreciated that the active agent is not limited to these applications. In various embodiments, the active agent is chosen from acne agents, antibiotics, antiseptics, antifungals, antibacterials, antimicrobials, biocides, anti-inflammatory, astringents, hormones, anticancer agents, smoking cessation compositions, cardiovasculars, histamine blockers, bronchodilators, analgesics, antiarrythmics, antihistamines, alpha-I blockers, beta blockers, ACE inhibitors, diuretics, antiaggregants, sedatives, tranquillizers, anticonvulsants, anticoagulant agents, vitamins, antiaging agents, agents for treating gastric and duodenal ulcers, anticellulites, proteolytic enzymes, healing factors, cell growth nutrients, peptides and others. Specific non-limiting examples of suitable active agents include penicillins, cephalosporins, tetracyclines, macrolides, epinephrine, amphetamines, aspirin, acetominophen, barbiturates, catecholamines, benzodiazepine, thiopental, codeine, morphine, procaine, lidocaine, benzocaine, sulphonamides, ticonazole, perbuterol, furosamide, prazosin, prostaglandins, salbutamol, indomethicane, diclofenac, glafenine, dipyridamole, theophylline and retinol. In one embodiment, the active agent is chosen from the group of coal tar, tazarotene, calcipotriene, calicineurin inhibitors, betamethasone, etanercept, adalimumab, infliximab, pimecrolimus, clobetasol propionate, glycyrrhetinic acid, zinc pyrithion, miconazole nitrate, zinc oxide, white petrolatum, alitretinoin, liarozole, bimosiamose, hydrocortisone, clobetasol, triamcinolone, fluocinonide, betamethasone, mometasone, desonide, alclometasone, diflorasone, amcinonide, pimecrolimus, tacrolimus, fuorate, metronidazol, tetracycline, and combinations thereof.

In various embodiments, the active agent is one or more of the following: scopolamine, nitroglycerin, clonidine, estradiol, fentanyl, nicotine, habitrol, testosterone, lidocaine, epinephrine, iontocaine, norethidrone, ethinyl estradiol, norelgestromin, levonorgestrel, oxybutynin, tetracaine, fentanyl HCl, methylphenidate, selegiline, rotigotine, rivastigmine, centella asiatica, retapamulin, alefacept, benzamycin, erythromycin, benzoyl peroxide, botulinum toxin type A, cefazolin, dextrose usp, chlorhexidine gluconate, clindamycin phosphate, pokofilox, desonide, adapalene gel, dynabac, elidel, norethindrone acetate, ketoconazole, azelaic acid, sodium sulfacet amide, terbinafine hydrochloride, betamethasone valerate, butenafine HCl, minoxidil, tacrolimus, becaplermin, tretinoin, ustekinumab, tigecycline, telavancin, levocetirizine dihydrochloride, niacinamide, ibuprofen, acetaminophen, aspirin, silver chloride, panthenol, clotrimazol, vitamin A, salicylic acid, and/or dexpanthenol. In another embodiment, the active agent is chosen from caffeine, lidocaine, and combinations thereof. It is also contemplated that the active agent may be chosen from lidocaine, niacinamide, ibuprofen, silver chloride, caffeine, and combinations thereof.

The active agent is present in the dispersion in an amount of from 0.01 to 20 weight percent based on a total weight of the dispersion. In various embodiments, the active agent is present in an amount of from 0.01 to 10, 0.5 to 20, 1 to 20, 2 to 18, 3 to 17, 4 to 16, 5 to 15, 6 to 13, 7 to 12, 8 to 11, or 9 to 10, weight percent based on the total weight of the dispersion. In other embodiments, the active agent is present in the dispersion in an amount of from 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, or 5 to 6, weight percent.

In the dispersion, the active agent is dispersed in the water independently from the plurality of encapsulated particles. In other words, the active agent is not dispersed in the core and/or layer disposed about the core of the particles of the first or second populations of encapsulated particles. Said differently, the active agent is dispersed in the water of the dispersion apart from the plurality of encapsulated particles. It is contemplated that the active agent may be added to the dispersion before, after, or simultaneously with the water and/or the first and/or second populations of encapsulated particles.

The active agent is also present in the film, as described in greater detail below, in an amount of from 0.02 to 60 weight percent based on a total weight of the film. In the film, the active agent is dispersed in the cured organopolysiloxane. In various embodiments, the active agent is present in the film in amounts of from 0.05 to 60, 1 to 60, 1 to 55, 5 to 50, 10 to 45, 15 to 40, 20 to 35, or 25 to 30, weight percent. In other embodiments, the active agent is present in an amount of from 0.5 to 20, 1 to 20, 2 to 18, 3 to 17, 4 to 16, 5 to 15, 6 to 13, 7 to 12, 8 to 11, or 9 to 10, weight percent, based on the total weight of a film.

### Physical Properties of the Dispersion

The dispersion of this disclosure is not limited to any particular physical properties. However, in various embodiments, the dispersion has a stability, maturation, and/or cure rate as described in PCT Ser. No. PCT/US10/40124 filed on Jun. 28, 2010, which is expressly incorporated herein by reference in its entirety.

Film (e.g. Transdermal Film):

The disclosure also provides a film, as first introduced above, and a method of forming the film. The film may be a transdermal film. Transdermal films are known in the art to be films applied to skin, typically unbroken skin. In one embodiment, transdermal refers to a route of administration of active agent delivered across the skin for systemic distribution. However, the film is not limited to such an embodiment and may be further defined as a one, two, or multilayer film. The film may be formed on skin or may be formed externally (e.g. on a substrate) and then applied to skin.

The film is not particularly limited relative to physical properties and may be alternatively described as a sheet. In various embodiments, the film has a thickness of from 0.0001 to 40 mils, from 0.1 to 1 mm, from 0.2 to 0.9 mm, from 0.3 to 0.8 mm, from 0.4 to 0.7 mm, from 0.5 to 0.6 mm, from 0.2 to 0.4 mm, from 0.01 to 0.1 mm, from 0.03 to 0.05 mm, etc. The film includes at least 40, 45, 50, 55, 60, 65, 70, 75, 80, 85, 90, 95, or 99, weight percent of a cured organopolysiloxane formed from the plurality of encapsulated particles. This weight percent is based on a total weight of the film. The cured organopolysiloxane includes the reaction product of the (a) first organopolysiloxane, the (d) second organopolysiloxane, and the (c) organohydrogensiloxane in the presence of the (b) hydrosilylation catalyst. The film also includes from 1 to 60 weight percent of the active agent dispersed in the cured organopolysiloxane. This weight percent is also based on a total weight percent of the film. The terminology “dispersed in” can include the active agent distributed in one or more portions of, or throughout, the cured organopolysiloxane.

The film is not particularly limited relative to stability, extractable fraction of unreacted organopolysiloxanes/organohydrogensiloxanes, maturation/cure rates, substantivity, vapor (e.g. water) permeability, sensory profiles, release rate and total release amount of the active agent(s), etc. as each of these properties may be customized based on application. However, in various embodiments, the film releases at least 80, 85, 90, 95, or 99, weight percent of the active agent to the skin or another suitable medium, as determined using an in-vitro release test across an artificial membrane as described in Brain K R, Walters A W, Watkinson A C, Method of Studying Percutaneous Absorption (Dermatological and Transdermal Formulations; Drug and the Pharmaceutical Sciences Series Vol. 119, Marcel Dekker Basel 2002, ISBN: 0824798899; pp. 197-296). In another embodiment, the film releases about 100 percent of the active agent to the skin. In other embodiments, the film has a substantivity of from 1 to 30, from 5 to 24, from 8 to 24, from 12 to 24, or from 18 to 24, hours without washing, as determined using the methods described in the Examples. Alternatively, the film can have a substantivity of greater than 24, 26, 48, or 60 hours, without washing. It is also contemplated that the film may have a substantivity after washing that is the same or different than the substantivity values described immediately above, as also determined using the methods described in the Examples. The film may also have a percent extractable of unreacted organopolysiloxanes/organohydrogensiloxanes of less than 20, 15, 10, or 5 percent after 5, 10, 15, 20, 30, 40, 45, 50, 60, 80, 90, 100, or 120 minutes of cure time, as determined using extraction and methods described in the Examples. In addition, the film may have one of the aforementioned percent extractable values after the plurality of encapsulated particles is aged for one or more of the following: 1 week at room temperature, 1 week at 45° C., 2 weeks at room temperature, 2 weeks at 45° C., 1 month at room temperature, 1 month at 45° C., 2 months at room temperature, and 2 months at 45° C., as determined using extraction and methods described in the Examples. In still other embodiments, the film has a vapor permeability (g/(hr*m²) of less than 160, 150, 140, 130, 120, 110, 100, 90, 80, 70, 60, etc., as determined using the Payne Cup test described in the Examples.

It is also contemplated that the film can be colored, opaque, transparent, translucent, etc. The film may be clear or transparent such that it is relatively unseen when disposed on skin. The film also is not particularly limited relative to dimensions such as length, width, or depth. Moreover, it is contemplated that more than one independent film can be disposed on one another. Alternatively, a single layer of the film can be utilized.

When used as a transdermal film, the film can be used to achieve a surface/aesthetic effect on skin and can be used to disperse fragrance. The transdermal film can also have a barrier or external effect to prevent movement of lice and other parasites across skin. The transdermal film can protect wounds and scars and can be customized for occlusivity. Alternatively, the transdermal film can exhibit an interface effect with keratin in/on skin and can have an emollient/hydration effect. In other embodiments, the transdermal film can topically deliver analgesics or other pain relievers to one or more layers of skin.

### Additional Components

In addition to (a)-(d) described above, the cores of any one or more of the encapsulated particles of the first and/or second populations, and/or the dispersion itself, may include one or more additional components. Similarly, the film itself may include one or more additional components to supplement those described above. These additional components may be silicone or organic components. In one embodiment, these components are substantially soluble with oil and substantially insoluble in water. Non-limiting examples of suitable additional components include silicones, such as volatile siloxanes, polydimethylsiloxane fluids, high molecular weight (i.e. molecular weight >1000) siloxanes, including silicone elastomers and resins, organic compounds such as, hydrocarbon oils, waxes, emollients, surfactants, thickeners, preservatives, antimicrobial, fragrances, colorants, colored indicators, diluents, extenders, excipients, pH buffers, stabilizers, preservatives, surfactants, fluorinated silicones, processing aids such as cyclic or linear polydiorganosiloxanes, bioadhesive materials, and/or hydrophilic, modulating and swellable components or polymers, such as those described in EP Publication 465,744, which is expressly incorporated herein by reference relative to these components and polymers. Other non-limiting examples include absorbents for wounds, alginate, polysaccharides, gelatin, collagen, and materials that can decrease friction. Still other non-limiting examples include absorbents, anticaking agents, antioxidants, antistatic agents, astringents, binders, buffering agents, bulking agents, chelating agents, astringents, deodorants, emollients, film formers, flavouring agents, humectants, lytic agents, moisturizing agents, occlusivity enhancers, opacifying agents, oxidizing and reducing agents, penetration enhancers, plasticizers, preservatives, bleaching agents, conditioning agents, protectants, slip modifiers, solubilizing agents, solvents, sunscreens, surface modifiers, surfactants and emulsifying agents, suspending agents, thickening agents, viscosity controlling agents, and UV light absorbers. Additional non-limiting examples of suitable additional components include alcohols, fatty alcohols and polyols, aldehydes, alkanolamines, alkoxylated alcohols (e.g. polyethylene glygol derivatives of alcohols and fatty alcohols), alkoxylated amides, alkoxylated amines, alkoxylated carboxylic acids, amides including salts (e.g. ceramides), amines, amino acids including salts and alkyl substituted derivatives, esters, alkyl substituted and acyl derivatives, polyacrylic acids, acrylamide copolymers, adipic acid copolymers, alcohols, aminosilicones, biological polymers and derivatives, butylene copolymers, carbohydrates (e.g. polysaccharides, chitosan and derivatives), carboxylic acids, carbomers, esters, ethers and polymeric ethers (e.g. PEG derivatives, PPG derivatives), glyceryl esters and derivatives, halogen compounds, heterocyclic compounds including salts, hydrophilic colloids and derivatives including salts and gums (e.g. cellulose derivatives, gelatin, xanthan gum, natural gums), imidazolines, inorganic materials (e.g. clay, TiO₂, ZnO), ketones (e.g. camphor), isethionates, lanolin and derivatives, organic salts, phenols including salts (e.g. parabens), phosphorus compounds (e.g. phosphate derivatives), polyacrylates and acrylate copolymers, protein and enzymes derivatives (e.g. collagen), synthetic polymers including salts, siloxanes and silanes, sorbitan derivatives, sterols, sulfonic acids and derivatives and waxes, salicylic acid, sulfur, calcium undecylenate, undecylenic acid, zinc undecylenate, povidone-iodine, alcohol, benzalkonium chloride, benzethonium chloride, hydrogen peroxide, methylbenzethonium chloride, phenol, poloxamer 188, acetyl cysteine, arbutin, ascorbic acid, ascorbic acid polypeptide, ascorbyl dipalmitate, ascorbyl methylsilanol pectinate, ascorbyl palmitate, ascorbyl stearate, BHA, p-hydroxyanisole, BHT, t-butyl hydroquinone, caffeic acid, camellia sinensis oil, chitosan ascorbate, chitosan glycolate, chitosan salicylate, chlorogenic acids, cysteine, cysteine HCl, decyl mercaptomethylimidazole, erythorbic acid, diamylhydroquinone, di-t-butylhydroquinone, dicetyl thiodipropionate, dicyclopentadiene/t-butylcresol copolymer, digalloyl trioleate, dilauryl thiodipropionate, dimyristyl thiodipropionate, dioleyl tocopheryl methylsilanol, isoquercitrin, diosmine, disodium ascorbyl sulfate, disodium rutinyl disulfate, di stearyl thiodipropionate, ditridecyl thiodipropionate, dodecyl gallate, ethyl ferulate, ferulic acid, hydroquinone, hydroxylamine hci, hydroxylamine sulfate, lsooctyl thioglycolate, kojic acid, madecassicoside, magnesium ascorbate, magnesium ascorbyl phosphate, melatonin, methoxy-PEG-7 rutinyl succinate, methylene di-t-butylcresol, methylsilanol ascorbate, nordihydroguaiaretic acid, octyl gallate, phenylthioglycolic acid, phloroglucinol, potassium ascorbyl tocopheryl phosphate, thiodiglycolamide, potassium sulfite, propyl gallate, rosmarinic acid, rutin, sodium ascorbate, sodium ascorbyl/cholesteryl, phosphate, sodium bisulfite, sodium erythorbate, sodium metabisulfide, sodium sulfite, sodium thioglycolate, sorbityl furfural, tea tree (melaleuca afiemifolia) oil, tocopheryl acetate, tetrahexyldecyl ascorbate, tetrahydrodiferuloylmethane, tocopheryl linoleate/oleate, thiodiglycol, tocopheryl succinate, thiodiglycolic acid, thioglycolic acid, thiolactic acid, thiosalicylic acid, thiotaurine, retinol, tocophereth-5, tocophereth-10, tocophereth-12, tocophereth-18, tocophereth-50, tocopherol, tocophersolan, tocopheryl linoleate, tocopheryl nicotinate, tocoquinone, o-tolyl biguanide, tris(nonylphenyl) phosphite, ubiquinone, and zinc dibutyldithiocarbamate, aluminium phenolsulfonate, ammonium phenolsulfonate, bakuchiol, benzalkonium bromide, benzalkonium cetyl phosphate, benzalkonium chloride, benzalkonium saccharinate, benzethonium chloride, potassium phenoxide, benzoxiquine, benzoxonium chloride, bispyrithione, boric acid, bromochlorophene, camphor benzalkonium methosulfate, captan, cetalkonium chloride, cetearalkonium bromide, cetethyldimonium bromide, cetrimonium bromide, cetrimonium chloride, cetrimonium methosulfate, cetrimonium saccharinate, cetrimonium tosylate, cetylpyridinium chloride, chloramine t, chlorhexidine, chlorhexidine diacetate, chlorhexidine digluconate, chlorhexidine dihydrochloride, p-chloro-m-cresol, chlorophene, p-chlorophenol, chlorothyrnol, chloroxylenol, chlorphenesin, ciclopirox olamine, climbazole, cloflucarban, clotrimazole, coal tar, colloidal sulfur, o-cymen-5-ol, dequalinium acetate, dequalinium chloride, dibromopropamidine diisethionate, dichlorobenzyl alcohol, dichlorophene, dichlorophenyl imidazoldioxolan, dichloro-m-xylenol, diiodomethyltolylsulfone, dimethylol ethylene thiourea, diphenylmethyl piperazinylbenzimidazole, domiphen bromide, 7-ethylbicyclooxazolidine, fluorosalan, formaldehyde, glutaral, hexachlorophene, hexamidine, hexamidine diisethionate, hexamidine diparaben, hexamidine paraben, hexetidine, hydrogen peroxide, hydroxymethyl dioxoazabicyclooctane, ichthammol, isopropyl cresol, lapyrium chloride, lauralkonium bromide, lauralkonium chloride, laurtrimonium bromide, laurtrimonium chloride, laurtrimonium trichlorophenoxide, lauryl isoquinolinium bromide, lauryl isoquinolinium saccharinate, laurylpyridinium chloride, mercuric oxide, methenamine, methenammonium chloride, methylbenzethonium chloride, myristalkonium chloride, myristalkonium saccharinate, myrtrimonium bromide, nonoxynol-9 iodine, nonoxynol-12 iodine, olealkonium chloride, oxyquinoline, oxyquinoline benzoate, oxyquinoline sulfate, PEG-2 coco-benzonium chloride, PEG-10 coco-benzonium chloride, PEG-6 undecylenate, PEG-8 undecylenate, phenol, o-phenylphenol, phenyl salicylate, piroctone olamine, sulfosuccinylundecylenate, potassium o-phenylphenate, potassium salicylate, potassium troclosene, propionic acid, pvp-iodine, quaternium-8, quaternium-14, quaternium-24, sodium phenolsulfonate, sodium phenoxide, sodium o-phenylphenate, sodium shale oil sulfonate, sodium usnate, thiabendazole, 2,2′-thiobis(4-chlorophenol), thiram, triacetin, triclocarban, triclosan, trioctyldodecyl borate, undecylenamidopropylamine oxide, undecyleneth-6, undecylenic acid, zinc acetate, zinc 30 aspartate, zinc borate, zinc chloride, zinc citrate, zinc cysteinate, zinc dibutyldithiocarbamate, zinc gluconate, zinc glutamate, zinc lactate, zinc phenolsulfonate, zinc pyrithione, zinc sulfate, and zinc undecylenate, benzyl alcohol, capsicum oleoresin (capsicum frutescens oleoresin), methyl salicylate, camphor, phenol, capsaicin, juniper tar (juniperus oxycedrus tar), phenolate sodium (sodium phenoxide), capsicum (capsicum frutescens), menthol, resorcinol, methyl nicotinate, and turpentine oil (turpentine), ammonium persulfate, calcium peroxide, hydrogen peroxide, magnesium peroxide, melamine peroxide, potassium bromate, potassium caroate, potassium chlorate, potassium persulfate, sodium bromate, sodium carbonate peroxide, sodium chlorate, sodium iodate, sodium perborate, sodium persulfate, strontium dioxide, strontium peroxide, urea peroxide, zinc peroxide, ammonium bisufite, ammonium sulfite, ammonium thioglycolate, ammonium thiolactate, cystemaine HCl, cystein, cysteine HCl, ethanolamine thioglycolate, glutathione, glyceryl thioglycolate, glyceryl thioproprionate, hydroquinone, p-hydroxyanisole, isooctyl thioglycolate, magnesium thioglycolate, mercaptopropionic acid, potassium metabisulfite, potassium sulfite, potassium thioglycolate, sodium bisulfite, sodium hydrosulfite, sodium hydroxymethane sulfonate, sodium metabisulfite, sodium sulfite, sodium thioglycolate, strontium thioglycolate, superoxide dismutase, thioglycerin, thioglycolic acid, thiolactic acid, thiosalicylic acid, zinc formaldehyde sulfoxylate, hydroquinone, allantoin, aluminium acetate, aluminium hydroxide, aluminium sulfate, calamine, cocoa butter, cod liver oil, colloidal oatmeal, dimethicone, glycerin, kaolin, lanolin, mineral oil, petrolatum, shark liver oil, sodium bicarbonate, talc, witch hazel, zinc acetate, zinc carbonate, zinc oxide, aminobenzoic acid, cinoxate, diethanolamine methoxycinnamate, digalloyl trioleate, dioxybenzone, ethyl 4-[bis(hydroxypropyl)] aminobenzoate, glyceryl aminobenzoate, homosalate, lawsone with dihydroxyacetone, menthyl anthranilate, octocrylene, octyl methoxycinnamate, octyl salicylate, oxybenzone, padimate 0, phenylbenzimidazole sulfonic acid, red petrolatum, sulisobenzone, titanium dioxide, trolamine salicylate, acetaminosalol, allatoin PABA, benzalphthalide, benzophenone, benzophenone 1-12,3-benzylidene camphor, benzylidenecamphor hydrolyzed collagen sulfonamide, benzylidene camphor sulfonic acid, benzyl salicylate, bornelone, bumetriozole, butyl methoxydibenzoylmethane, butyl PABA, ceria/silica, ceria/silica talc, cinoxate, DEA-methoxycinnamate, dibenzoxazol naphthalene, di-t-butyl hydroxybenzylidene camphor, digalloyl trioleate, diisopropyl methyl cinnamate, dimethyl PABA ethyl cetearyldimonium tosylate, dioctyl butamido triazone, diphenyl carbomethoxy acetoxy naphthopyran, disodium bisethylphenyl tiamminotriazine stilbenedisulfonate, disodium distyrylbiphenyl triaminotriazine stilbenedisulfonate, disodium distyrylbiphenyl disulfonate, drometrizole, drometrizole trisiloxane, ethyl dihydroxypropyl PABA, ethyl diisopropylcinnamate, ethyl methoxycinnamate, ethyl PABA, ethyl urocanate, etrocrylene ferulic acid, glyceryl octanoate dimethoxycinnamate, glyceryl PABA, glycol salicylate, homosalate, isoamyl pmethoxycinnamate, isopropylbenzyl salicylate, isopropyl dibenzolylmethane, isopropyl methoxycinnamate, menthyl anthranilate, menthyl salicylate, 4-methylbenzylidene, camphor, octocrylene, octrizole, octyl dimethyl PABA, octyl methoxycinnamate, octyl salicylate, octyl triazone, PABA, PEG-25 PABA, pentyl dimethyl PABA, phenylbenzimidazole sulfonic acid, polyacrylamidomethyl benzylidene camphor, potassium methoxycinnamate, potassium phenylbenzimidazole sulfonate, red petrolatum, sodium phenylbenzimidazole sulfonate, sodium urocanate, tea-phenylbenzimidazole sulfonate, tea-salicylate, terephthalylidene dicamphor sulfonic acid, titanium dioxide, tripaba panthenol, urocanic acid, and VA/crotonates/methacryloxybenzophenone-1 copolymers. To the extent that one or more of the aforementioned compounds may also be a pharmaceutically active agent, such as a drug, therapeutic agent, etc., it is contemplated that such a compound could alternatively be utilized as the active agent in this disclosure.

One or more of these additional components may be utilized in amounts as described in PCT Ser. No. PCT/US10/40124 filed on Jun. 28, 2010, which is expressly incorporated herein by reference in its entirety. In various embodiments, one or more additional components may be present in an amount of up to 50 wt percent of a weight of the plurality of encapsulated particles and/or of a weight of the film.

### Method of Forming the Film:

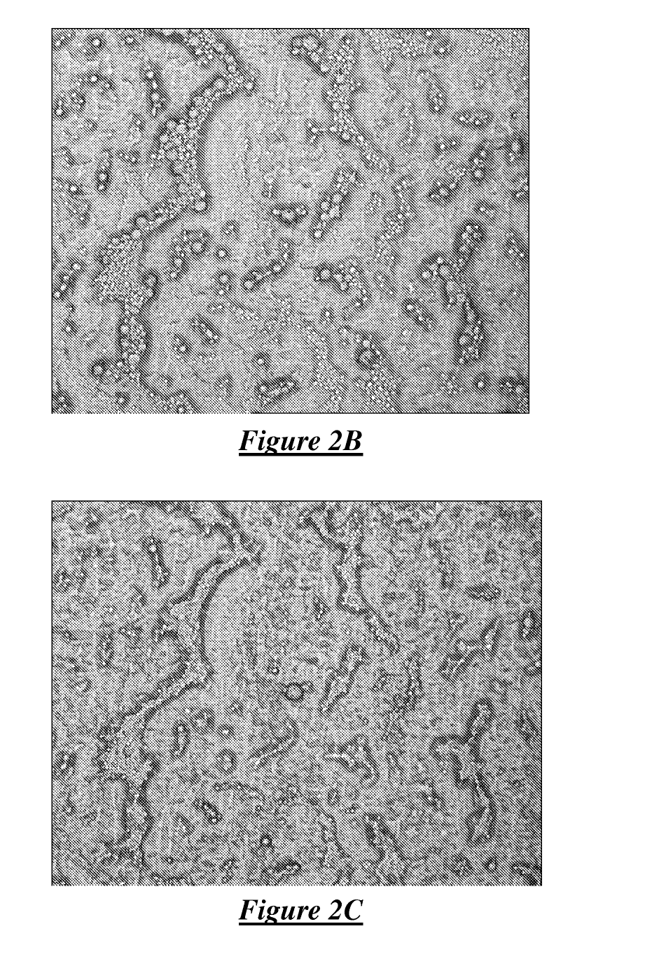

The method of forming the film may include the steps of applying the dispersion to a substrate (such as a release liner) or to skin (e.g. mammalian or human skin) and allowing the water to evaporate. Alternatively, the dispersion may be heated to expedite evaporation of the water. It is also contemplated that one or more burst aids may be used. The goal of evaporating the water and/or using the burst aids is to maximize aggregation, coalescence, and breakage/bursting of the plurality of encapsulated particles (see FIGS. 2A-2C) to allow the (a) first organopolysiloxane and (d) second organopolysiloxane to react with the (b) organohydrogensiloxane in the presence of the (b) hydrosilylation catalyst and cure to form a cured organopolysiloxane having an amount of the active agent disposed therein. The water may evaporate in a time of less than 10, 5, 4, 3, 2, or 1 minute(s).

### EXAMPLES

A series of active agent delivery dispersions (Dispersions 1-3) are formed according to this disclosure. Dispersion 1 includes a first population of encapsulated particles wherein the core includes a vinyl PDMS and a platinum catalyst and a second population of encapsulated particles wherein the core includes the same vinyl PDMS and an SiH siloxane. The plurality of encapsulated particles in Dispersion 1 is present in about 30 weight percent in water. Dispersion 2 is further defined as Dispersion 1 that is diluted to about 5 wt % of the encapsulated particles in water. Dispersion 3 is further defined as Dispersion 1 that is diluted to about 1 wt % of the encapsulated particles in water. Notably, none of Dispersions 1-3, at least initially, includes an active agent.

### Extractable Percent of Unreacted Silicone—First Evaluation

Various samples of Dispersion 1 are aged for the following times and at the following temperatures to age the encapsulated particles therein: 1 week at room temperature, 1 week at 45° C., 2 weeks at room temperature, 2 weeks at 45° C., 1 month at room temperature, 1 month at 45° C., 2 months at room temperature, and 2 months at 45° C.

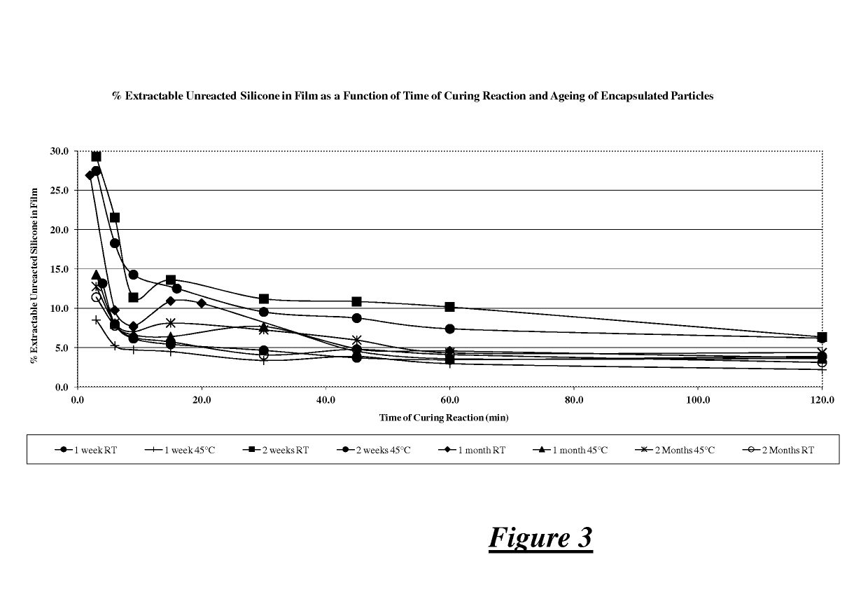

After ageing, the samples are spread out onto silicon free Glassine paper and the water is allowed to evaporate such that the vinyl PDMS and SiH siloxane reaction and form films. More specifically, about 2 grams of each sample is spread using a 1.2 g/m² bar equipped with an automatic film application (type 4340 from Braive Instruments). At times from zero to 120 minutes, the percent of vinyl PDMS and SiH siloxane is determined using 30 ml of methylisobutyl ketone (MIBK). Additionally, silicone concentration on the Glassine paper resulting from reacted vinyl PDMS and SiH siloxane is determined by XRF using an Oxford Lab X-3000. The percent unreacted/extractable vinyl PDMS and SiH siloxane is plotted in an extractable plot set forth as FIG. 3.

The results of these evaluations indicate that ageing affects a total amount of vinyl PDMS and SiH siloxane that react/fail to react. However, in each evaluation, the total amount of vinyl PDMS and SiH siloxane that do not react, at the end of 120 minutes, is approximately the same low amount indicating that the film is still very reactive after ageing.

### Extractable Percent of Unreacted Silicone—Second Evaluation

Additional samples of Dispersion 1 are aged for 1 week at 45° C. After ageing various amounts of one of caffeine, lidocaine, vitamin B3, clotrimazol, salicylic acid, dexpanthenol, and AgCl (as examples of active agents) are added to individual samples. At this point, films are formed from these samples to determine an effect of the active agents on curing and reaction of the vinyl PDMS and SiH siloxane. More specifically, for these samples, an additional extractable plot is generated (not shown in the Figures) using the same method as described above. Subsequently, cure rate (i.e., reactivity) is determined after 3 minutes and after 120 minutes by measuring the slope of the extractable plot after coating of the Glassine paper. The results of these evaluations are set forth in Table 1 immediately below with lower values representing higher reactivity.

TABLE 1

| | Concentration | | |
|---|---|---|---|
| | (%) of Active | Cure Rate | Cure Rate |
| | Agents in | After | After |
| | Samples of | 3 Min. | 120 Min. |
| Active Agents | Dispersion 1 | (%) | (%) |
| Caffeine | 1 | 31.7 | 8.5 |
| Lidocaine | 1 | 21.1 | 4.9 |
| Vitamin B3 | 1 | 43.3 | 21.8 |
| Clotrimazol | 1 | 25.7 | 5.4 |
| Salicylic acid | 5 | 36.5 | 12.1 |
| Dexpanthenol | 2.5 | 18.4 | 4.8 |
| AgCl | 0.5 | 16.4 | 3.3 |

These results indicate that, for most active agents, the reactivity after 120 minutes is approximately the same ±about 3%. However, the reactivity of the vinyl PDMS and SiH siloxane in the samples that include the Vitamin B3 and the Salicylic acid are higher. This indicates slight inhibition of the cure but still in an acceptable range.

### The Payne Cup Test

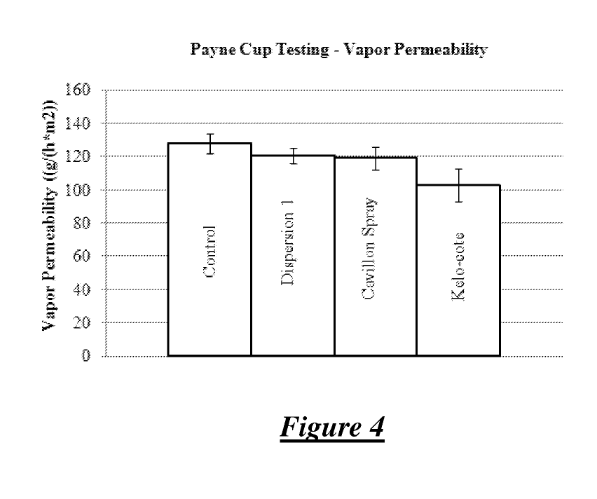

As is known, moisturization of skin is accomplished, at least in part, by increasing its water content. This can be done by occlusion, which prevents the loss of water vapor from the skin. A film formed from Dispersion 1 is evaluated using a Payne Cup Test to evaluate the occlusivity of the film. Two additional films formed from Cavillon and Kelo-cote, each of which is a commercially available product used to treat scars or protect the skin, are also evaluated. More specifically, the Payne Cup Test utilizes a collagen membrane placed over a stainless steel cup loaded with water, in a controlled temperature environment. Water weight loss over time gives a rate of evaporation that can be directly compared to transepidermal water loss (TEWL) as measured by an evaporimeter. In this case, a control collagen membrane with no film disposed thereon is evaluated along with collagen membranes that each independently have the film formed from Dispersion 1, the film formed from Cavillon, or the film formed from Kelo-cote, disposed thereon. The results of the Payne Cup Test are set forth in the graph of FIG. 4 and indicate that the film formed from Dispersion 1 is not occlusive to the skin like Cavillon and Kelo-cote. This occlusivity is higher for the film of this disclosure than for the films formed from Cavillon and Kelo-cote.

Substantivity vs. Washes

Films formed from Dispersion 1 and Dispersion 2 and films formed from Cavillon and Kelo-cote, as described above, are also evaluated to determine substantivity as compared to a number of times the films are “washed” on human skin. The substantivity of the films is evaluated to measure a long lasting effect (i.e., “staying” ability) of the films, which is detectable by infrared spectroscopy.

More specifically, samples of Dispersions 1 and 2 and samples of Cavillon and Kelo-cote are placed on the skin of four human subjects in approximately identical amounts to form films of approximately the same size and thickness. The substantivity of the films is assessed before washing, after a first wash, after a second wash, and after a third wash. Each wash is completed by wetting the skin for approximately 5 seconds using 37° C. tap water and then applying approximately 3 ml of a 0.5% SLES solution to the skin and rubbing 15 times over the test site. The substantivity is quantified using a FT-infrared (FTIR) Perkin Elmer Spectrum One Spectrometer equipped with a ZnSe 45° (flat plate) HATR sampling accessory. The FTIR parameters include a resolution of 2 cm⁻¹, 16 background scans, and a spectral range of 4000 cm⁻¹ to 650 cm⁻¹.

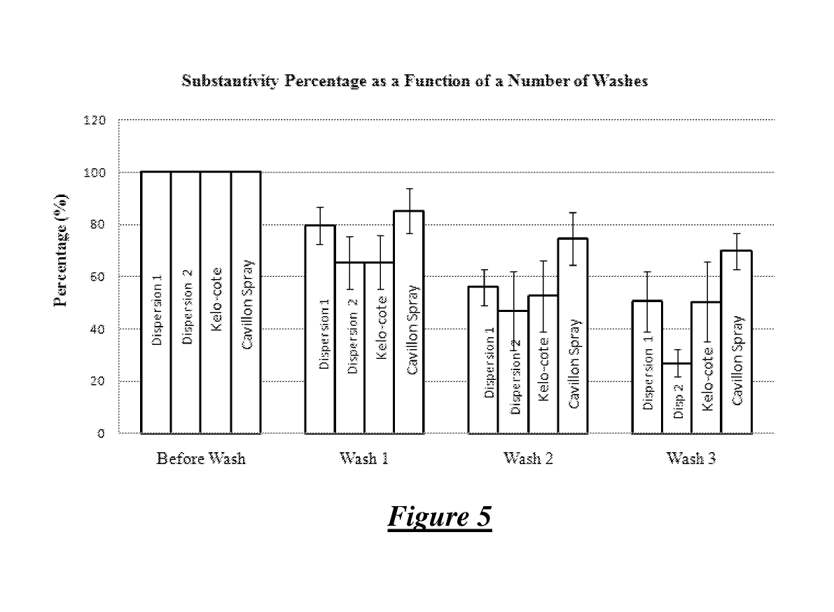

The results of these evaluations are set forth in FIG. 5 and indicate that the transdermal film formed from Dispersion 1 provides good substantivity versus washes wherein about 50% of the film remains on the skin after 3 washes. The results also indicate that the transdermal film formed from Dispersion 1 provides good substantivity after 1 wash wherein about 65% of the film remains on the skin and moderate substantivity after 3 washes wherein about 30% of the film remains on the skin. The results also indicate that the transdermal film formed from Dispersion 2 is slightly less substantive after the washes. Moreover, the results indicate that the film of this disclosure has the same resistance to water as the currently commercially available products.

In addition, when the Dispersions 1 and 2 are evaluated apart from the skin, the amide peak in the FT-IR spectra (not shown in the Figures) is reduced and appears as a very small peak. In addition, a large silicone peak is shown. This suggests that the transdermal film formed from Dispersions 1 and 2 have good homogeneity and are formed with appropriate thicknesses.

Substantivity vs. Time

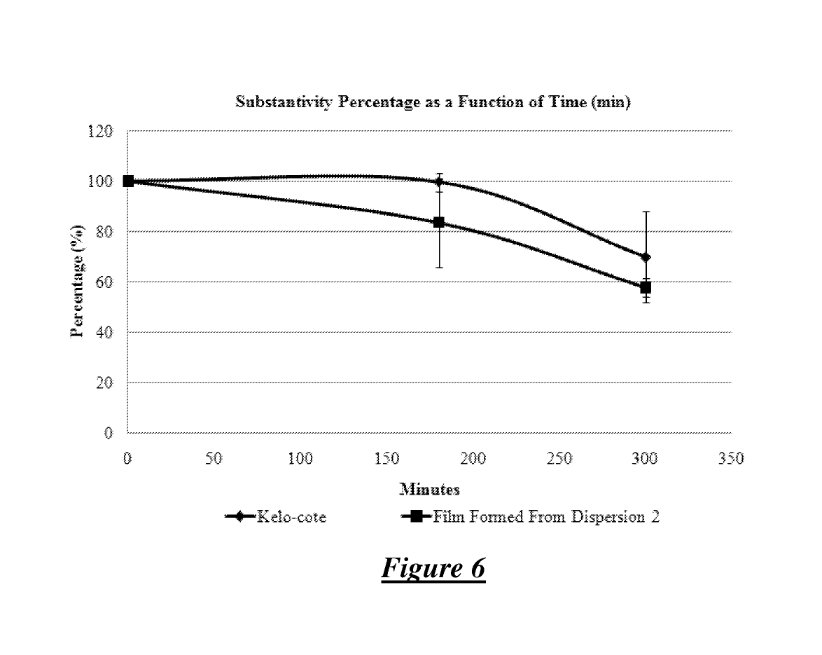

A film formed from Dispersion 2 is compared against a film formed from Kelo-cote (which is commercially available from Advanced Bio-Technologies, Inc. as an active agent delivery composition) that is diluted to 5 wt % in isodecane. These films are evaluated in the same way as described immediately above except that no washes are utilized. The results of these evaluations are set forth in FIG. 6 and indicate that the film formed from Dispersion 2 has a similar substantivity profile as the Kelo-cote.

### Sensory Profile Evaluation

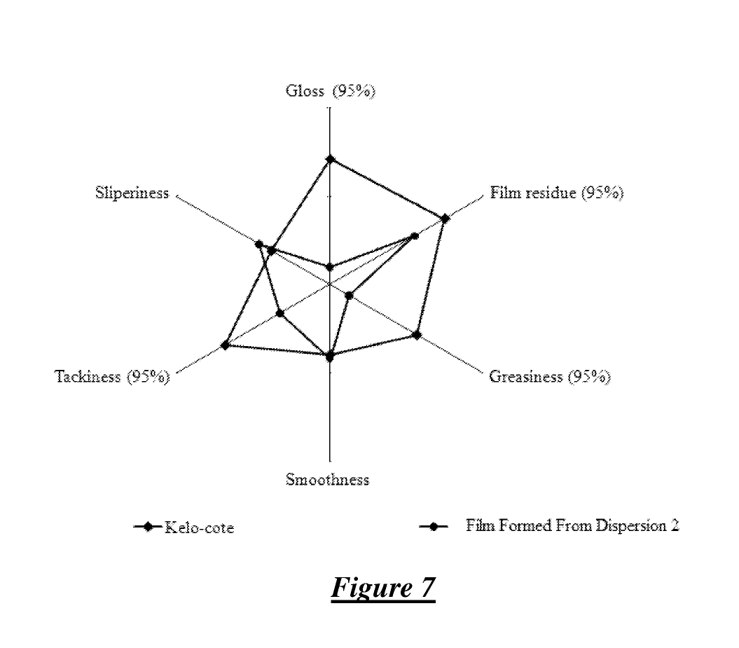

An additional film formed from Dispersion 2 and an additional film formed from Kelo-cote (described above) are each evaluated to determine tackiness, gloss, residue, grease, slipperiness, and smoothness. More specifically, these films are formed in approximately the same size and thickness on the skin of four human subjects. Tackiness is generally assessed using a finger and the subjects determine non-tackiness as compared to a mineral oil standard and tackiness as compared to a lanolin standard. Gloss is generally assessed based on a perceived amount of light reflected off skin. High gloss is determined as compared to a mineral oil standard. Low gloss is determined as compared to a talc standard. Residue is generally assessed based on an amount of the transdermal film remaining on the skin after drying of the film. High residue is determined as compared to a petrolatum standard. Low residue is determined as compared to an untreated skin standard. Grease is generally assessed based on the perceived presence of a tacky, dense coating that includes drag. Low grease is determined as compared to a talc standard. High grease is determined as compared to a petrolatum standard. Slipperiness is generally assessed based on an ease of moving fingers across the transdermal films. High slipperiness (easy to move finger across skin) is determined as compared to a mineral oil standard. Low slipperiness (difficult to move finger across skin) is determined as compared to a lanolin standard. Smoothness is generally assessed based on a perceived evenness of the transdermal films and their uniformity of texture. Roughness (i.e., low smoothness) is determined as compared to a gritty cleanser standard. High smoothness is determined as compared to a talc standard. The results of these evaluations are averaged and summarized in FIG. 7 and indicate that the film formed from Dispersion 1 is significantly less greasy and less tacky than the film formed from Kelo-cote.

### Cumulative Lidocaine Delivery Across Pig Skin

To evaluate cumulative delivery of lidocaine, 1 gram of lidocaine is combined with 99 grams of a sample of Dispersion 3 which is used to form a film. As a comparative example, a film is also formed from EMLA which is a 5% dispersion including 2.5% each of lidocaine/prilocalne and is marketed by APP Pharmaceuticals as abbreviation for Eutectic Mixture of Local Anesthetics (EMLA).

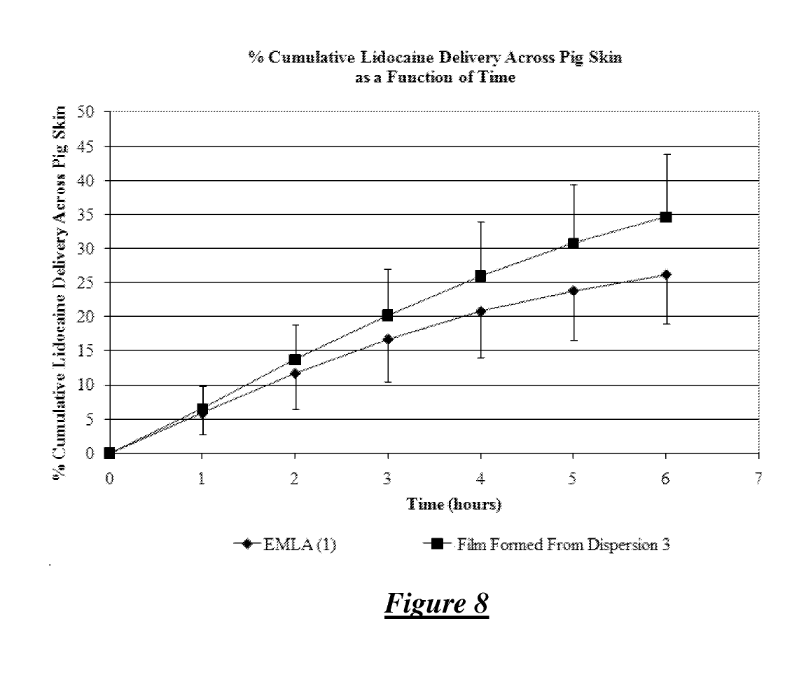

After formation, the films are evaluated using a Franz static diffusion test. This test is designed to evaluate the percutaneous permeation of active agents (such as lidocaine and caffeine) through/across pig-skin. As is known, permeation of active agents through skin can be affected by method of delivery. The Franz static diffusion test utilizes a pig-skin membrane upon which the film formed from Dispersion 3 is disposed and also utilizes a pig-skin membrane upon which the film formed from EMLA is disposed. In essence, the Franz static diffusion test measures whether the film formed from Dispersion 3 performs as well or better than the film formed from the EMLA relative to delivery of the lidocaine across the pig skin. The results of these evaluations are set forth in FIG. 8 and indicate that the film formed from Dispersion 3 has superior performance as the film formed from the EMLA.

### Cumulative Caffeine Delivery Across Pig Skin

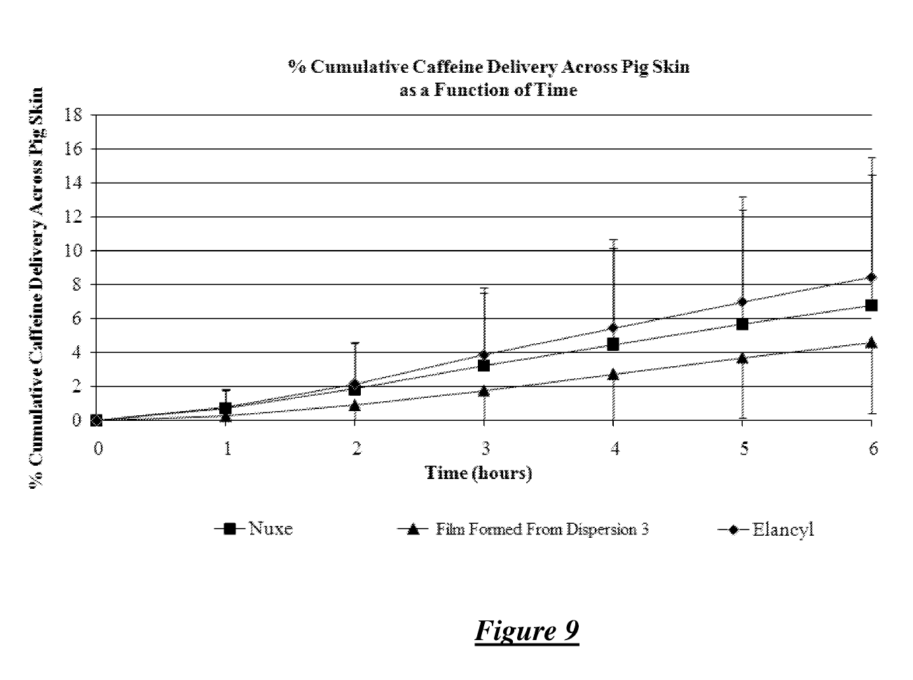

To evaluate cumulative delivery of caffeine, 1 gram of caffeine is combined with 99 grams of a sample of Dispersion 3. As a first comparative example, 2 grams of caffeine are included in 100 grams of a sample of Nuxe, which is a commercially available product used to delivery caffeine to skin. As a second comparative example, 2.6 grams of caffeine are included with 100 grams of a sample of Elancyl, which is also a commercially available product used to deliver caffeine to skin. Each of the Dispersion 3, the Nuxe, and the Elancyl are used to form films using the same method as described above. The same Franz static diffusion test, as described above, is employed to evaluate delivery of caffeine across the pig skin. The results of these evaluations are set forth in FIG. 9 and indicate that the film formed from Dispersion 3 has about the same performance as the film formed from the Nuxe and the Elancyl. Relative to absolute amounts, the film formed from Dispersion 3 delivers less caffeine because the formulation contains only 1% while the benchmarks have 2 and 2.6% caffeine, respectively. Compared to the amount applied, all formulations deliver the same amount of caffeine. Accordingly, the data indicates that the film formed from Dispersion 3 performs similarly as the commercially available products.

In sum, the aforementioned results demonstrate that the films of this disclosure perform approximately as well or even better than many commercial products. However, the results are surprising in that the films of this disclosure produce unexpected and superior results relative to non-occlusivity, substantivity, water resistance, skin delivery, and overall sensory profile. These results are summarized immediately below:

| | | | | | Accept- |
|---|---|---|---|---|---|
| | | Acceptable | Effective | Effective | able |
| | Non- | Sub- | Water | Skin | Sensory |
| | Occlusivity | stantivity | Resistance | Delivery | Profile |
| EMLA | | | | Yes | |
| Cavillon | Yes | Yes | Yes | | Yes |
| Kelo-cote | Yes | Yes | Yes | | Yes |
| Nuxe | | | | Yes | |
| Elancyl | | | | Yes | |
| Disclosure | Yes | Yes | Yes | Yes | Yes |

### Cumulative Ibuprofen Delivery Across Pig Skin

To evaluate cumulative delivery of ibuprofen, 1 gram of ibuprofen is combined with 59 grams of a sample of Dispersion 1, which is used to form a film, and 40 g of propylene glycol. As a comparative example, a film is also formed from “Ibutop Gel” which is a commercially available product used to deliver ibuprofen to skin and which includes 5 wt % of ibuprofen.

The same Franz static diffusion test, as described above, is used to evaluate delivery of ibuprofen across the pig skin. The results of these evaluations indicate that the film formed from Dispersion 1 has about the same performance as the film formed from the Ibutop gel. Relative to absolute amounts, the film formed from Dispersion 1 delivers less ibuprofen because the formulation includes only 1 wt % while the Ibutop Gel include 5 wt % ibuprofen. When directly compared to the amount applied, the film formed from Dispersion 1 delivers a larger amount of ibuprofen. Accordingly, the data indicates that the film formed from Dispersion 1 has superior performance to the film formed from the Ibutop Gel.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is to be understood that any value or range of values described above may vary by ±5%, ±10%, ±15%, ±20%, ±25%, ±30%, etc. It is also to be understood that any range of values may be further defined as any range of whole and/or fractional values therein. It is further to be understood that any ranges and subranges relied upon in describing various embodiments of the present disclosure independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present disclosure, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

1. An active agent delivery dispersion comprising:
A. water;
B. 1 to 98 weight percent of a plurality of encapsulated particles based on a total weight of said dispersion, wherein said encapsulated particles are dispersed in the water, wherein each of said particles comprises a core and a layer comprising a silica that is disposed about said core, and wherein said plurality comprises;
(i) a first population of encapsulated particles wherein said core comprises,
(a) a first organopolysiloxane having at least two silicon-bonded alkenyl groups per molecule, and
(b) a hydrosilylation catalyst, and
(ii) a second population of encapsulated particles wherein said core comprises,
(c) an organohydrogensiloxane having an average of greater than two silicon bonded hydrogen atoms per molecule, and
(d) a second organopolysiloxane having at least two silicon-bonded alkenyl groups per molecule that is the same or different from said (a) first organopolysiloxane; and
C. 0.01 to 20 weight percent of an active agent based on a total weight of said dispersion wherein said active agent is dispersed in said water independently from said plurality of encapsulated particles.

2. A dispersion as set forth in claim 1 wherein said silicon bonded hydrogen atoms of said (c) organohydrogensiloxane are present in a molar ratio of about 1 to about 4 with said silicon-bonded alkenyl groups of said (a) first organopolysiloxane.

3. A dispersion as set forth in claim 1 wherein said silicon bonded hydrogen atoms of component (c) are present in a molar ratio of about 3 to about 10 with said silicon-bonded alkenyl groups of said (a) first organopolysiloxane.

4. A dispersion as set forth in claim 1 wherein said (a) first organopolysiloxane comprises at least two siloxane units per molecule that each independently have the average formula R²RₘSiO_{(4-m)/2}, wherein
each R is independently a hydrocarbon group having from 1 to 20 carbon atoms,
each R² is a monovalent alkenyl aliphatic group, and
m is a number of from 0 to 2.

5. A dispersion as set forth in claim 1 wherein said (a) first organopolysiloxane has an average formula that is further defined as:
CH₂═CH(Me)₂SiO[Me₂SiO]_{x′}Si(Me)₂CH═CH₂;
CH₂—CH—(CH₂)₄-(Me)₂SiO[Me₂SiO]_{x′}Si(Me)₂-(CH₂)₄—CH═CH₂; or
Me₃SiO[(Me)₂SiO]_{x′}[CH₂═CH(Me)SiO]_{x″}SiMe₃,
and wherein Me is methyl, x′≧0, and x″≧2.

6. A dispersion as set forth in claim 1 wherein said (c) organohydrogensiloxane has an average formula (R³₃SiO_{1/2})ₐ(R⁴₂SiO_{2/2})_{b}(R⁴HSiO_{2/2})_{c}, wherein
each R³ is independently a hydrogen atom or R⁴,
each R⁴ is independently a monovalent hydrocarbyl having 1 to 10 carbon atoms, and wherein a≧2, b≧0, and c≧2.

7. A dispersion as set forth in claim 1 wherein said (c) organohydrogensiloxane is further defined as a dimethyl, methyl-hydrogen polysiloxane having an average formula (CH₃)₃SiO[(CH₃)₂SiO]_{b}[(CH₃)HSiO]_{c}Si(CH₃)₃, wherein b≧0, and c≧2.

8. A method of forming the dispersion of claim 1.

9. A method as set forth in claim 8 comprising the steps of:
A. forming a first oil phase comprising the (a) organopolysiloxane and the (b) hydrosilylation catalyst,
B. combining the first oil phase with an aqueous phase comprising a surfactant to form an oil in water emulsion having an oil/water interface,
C. adding a tetraalkoxysilane to the oil in water emulsion,
D. polymerizing the tetraalkoxysilane at the oil/water interface of the emulsion to form the first population of encapsulated particles wherein the layer disposed about the core of the first population of encapsulated particles is silica,
E. forming a second oil phase comprising the (c) organohydrogensiloxane, and the (d) second organopolysiloxane,
F. repeating steps B-D to form the second population of encapsulated particles wherein the layer disposed about the core of the second population of encapsulated particles is silica, and
G. combining the first and second populations of the encapsulated particles to form the dispersion.

10. A film comprising:
A. at least 40 weight percent of a cured organopolysiloxane based on a total weight of said film wherein said cured organopolysiloxane is formed from a plurality of encapsulated particles wherein each of said particles comprises a core and a layer comprising a silica that is disposed about said core, and wherein said plurality comprises:
(i) a first population of encapsulated particles wherein said core comprises,
(a) a first organopolysiloxane having at least two silicon-bonded alkenyl groups per molecule, and
(b) a hydrosilylation catalyst, and
(ii) a second population of encapsulated particles wherein said core comprises,
(c) a organohydrogensiloxane having an average of greater than two silicon bonded hydrogen atoms per molecule, and
(d) a second organopolysiloxane having at least two silicon-bonded alkenyl groups per molecule that is the same or different from said (a) first organopolysiloxane;
wherein said cured organopolysiloxane comprises the reaction product of said (a) first organopolysiloxane, said (d) second organopolysiloxane, and said (c) organohydrogensiloxane in the presence of said (b) hydrosilylation catalyst; and
B. 0.02 to 60 weight percent of an active agent based on a total weight of said film wherein said active agent is dispersed in said cured organopolysiloxane.

11. A film as set forth in claim 10 comprising at least 90 weight percent of said cured organopolysiloxane based on a total weight of said film and comprising 1 to 6 weight percent of said active agent.

12. A film as set forth in claim 11 wherein said silicon bonded hydrogen atoms of said (c) organohydrogensiloxane are present in a molar ratio of about 1 to about 4 with said silicon-bonded alkenyl groups of said (a) first organopolysiloxane.

13. A film as set forth in claim 11 wherein said silicon bonded hydrogen atoms of component (c) are present in a molar ratio of about 3 to about 10 with said silicon-bonded alkenyl groups of said (a) first organopolysiloxane.

14. A film as set forth in claim 10 wherein said (a) first organopolysiloxane comprises at least two siloxane units per molecule that each independently have the average formula R²RₘSiO_{(4-m)/2}, wherein
each R is independently a hydrocarbon group having from 1 to 20 carbon atoms,
each R² is a monovalent alkenyl aliphatic group, and
m is a number of from 0 to 2.

15. A film as set forth in claim 10 wherein said (a) first organopolysiloxane has an average formula that is further defined as:
CH₂═CH(Me)₂SiO[Me₂SiO]_{x′}Si(Me)₂CH═CH₂;
CH₂═CH—(CH₂)₄-(Me)₂SiO[Me₂SiO]_{x′}Si(Me)₂-(CH₂)₄—CH═CH₂; or
Me₃SiO[(Me)₂SiO]_{x′}[CH₂═CH(Me)SiO]_{z″}SiMe₃,
and wherein Me is methyl, x′≧0, and x″≧2.

16. A film as set forth in claim 10 wherein said (c) organohydrogensiloxane has an average formula (R³₃SiO_{1/2})ₐ(R⁴₂SiO_{2/2})_{b}(R⁴HSiO_{2/2})_{c}, wherein
each R³ is independently a hydrogen atom or R⁴,
each R⁴ is independently a monovalent hydrocarbyl having 1 to 10 carbon atoms, and wherein a≧2, b≧0, and c≧2.

17. A film as set forth in claim 10 wherein said (c) organohydrogensiloxane is further defined as a dimethyl, methyl-hydrogen polysiloxane having an average formula (CH₃)₃SiO[(CH₃)₂SiO]_{b}[(CH₃)HSiO]_{c}Si(CH₃)₃, wherein b≧0, and c≧2.

18. A film as set forth in claim 10 that is further defined as a transdermal film.

19. A method of forming a film comprising at least 40 weight percent of a cured organopolysiloxane based on a total weight of the film and 1 to 60 weight percent of an active agent based on a total weight of the film, wherein the active agent is dispersed in the cured organopolysiloxane and wherein said method comprises the step of applying a dispersion to a substrate to form the film, the dispersion comprising:
A. water;
B. 1 to 98 weight percent of a plurality of encapsulated particles based on a total weight of the dispersion, wherein the encapsulated particles are dispersed in water, each of the particles comprises a core and a layer comprising a silica that is disposed about the core, and the plurality comprises;
(i) a first population of encapsulated particles wherein the core comprises,
(a) a first organopolysiloxane having at least two silicon-bonded alkenyl groups per molecule, and
(b) a hydrosilylation catalyst, and
(ii) a second population of encapsulated particles wherein the core comprises,
(c) an organohydrogensiloxane having an average of greater than two silicon bonded hydrogen atoms per molecule, and
(d) a second organopolysiloxane having at least two silicon-bonded alkenyl groups per molecule that is the same or different from the (a) first organopolysiloxane; and
C. 0.1 to 20 weight percent of an active agent based on a total weight of the dispersion wherein the active agent is dispersed in the water independently from the plurality of encapsulated particles.

20. A method as set forth in claim 19 wherein the film comprises at least 90 weight percent of the cured organopolysiloxane based on a total weight of the film and comprises 1 to 6 weight percent of the active agent.

21. A method as set forth in claim 20 further comprising the step of bursting one or more encapsulated particles from both the first and second populations such that the (i) first organopolysiloxane and the (iv) second organopolysiloxane react with the (iii) organohydrogensiloxane in the presence of the (ii) hydrosilylation catalyst to form the cured organopolysiloxane.

22. A method as set forth in claim 21 wherein the step of bursting is further defined as adding a burst aid to the dispersion.

23. A method as set forth in claim 21 wherein the step of bursting is further defined as heating the dispersion.

24. A method as set forth in claim 19 wherein the substrate is further defined as skin, the film is further defined as a transdermal film, and the (i) first organopolysiloxane comprises at least two siloxane units per molecule that each independently have the average formula R²RₘSiO_{(4-m)/2}, wherein
each R is independently a hydrocarbon group having from 1 to 20 carbon atoms,
each R² is a monovalent alkenyl aliphatic group, and
m is a number of from 0 to 2, and
wherein the (iii) organohydrogensiloxane has an average formula (R³₃SiO_{1/2})ₐ(R⁴₂SiO_{2/2})_{b}(R⁴HSiO_{2/2})_{c}, wherein
each R³ is independently a hydrogen atom or R⁴,
each R⁴ is independently a monovalent hydrocarbyl having 1 to 10 carbon atoms, and wherein a≧2, b≧0, and c≧2.
